(19) United States
(12) Patent Application Publication
(10) Pub. No.: US 2010/0184603 A1
(43) Pub. Date: Jul. 22, 2010

(54) Alcohol alkoxylates, compositions containing these, and use of the alcohol alkoxylates as adjuvants for the agrochemical field

(75) Inventors: Michael Stoesser, Neuhofen (DE); Rainer Berghaus, Speyer (DE); Gerhard Krennrich, Frankenthal (DE); Markus Kummeter, Heddesheim (DE); Guenter Oetter, Frankenthal (DE); Ulrich Steinbrenner, Neustadt (DE); Norbert Wagner, Mutterstadt (DE); Peter Dombo, Wiesbaden (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/597,043

(22) PCT Filed: Apr. 24, 2008
(86) PCT No.: PCT/EP08/55034

§ 371 Date: Feb. 19, 2010

(30) Foreign Application Priority Data
Apr. 25, 2007 (EP) 07106961.1

(51) Int. Cl. C07C 43/11; C07C 31/18; A01N 25/02; A01P 13/00

(52) U.S. Cl. 504/362; 568/852; 568/680

(57) ABSTRACT

The present invention relates to certain alcohol alkoxylates of the amphiphilic type, to agrochemical compositions comprising them, and to the use of the alcohol alkoxylates as activity-enhancing adjuvant in the agrochemical sector and in particular in the sector of plant protection. The alcohol alkoxylates are alkoxylated alcohols of the formula (I)

R—O—(CₘH₂ₘO)_{q}—[(C₂H₄O)_{z}—(CₙH₂ₙO)ₓ—(C₂H₄O)_{y}]_{co}—Z (I)

in which
R represents C₁-C₇-alkyl;
m represents 2 or 3;
q represents 0, 1, 2 or 3;
n represents an integer from 3 to 16;
x is a value of from 1 to 100;
y is a value of from 0 to 100;
z is a value of from 0 to 100; and
x+y+z corresponds to a value of from 2 to 100;
Z represents hydrogen or an end group cap,
   where at least one of y and z is greater than 0.

The present invention relates to certain alcohol alkoxylates of the amphiphilic type, agrochemical compositions comprising these, and the use of the alcohol alkoxylates as activity-improving adjuvant in the agrochemical sector, and in particular in the plant protection sector.

Besides the optimization of the active ingredient properties, the development of an effective agent is of particular importance with regard to the industrial production and application of active ingredients. By formulating the active ingredient(s) correctly, an optimal balance must be found between properties, some of which are in conflict with each other, such as the biological activity, the toxicology, potential environmental effects, and the costs. Moreover, the formulation is a decisive factor in determining the shelf life and the user friendliness of a composition.

An efficient uptake of the active ingredient by the plant is of particular importance for the activity of an agrochemical composition. If this uptake is via the leaf, it constitutes a complex translocation process, where the active substance, for example a herbicide, must first penetrate the waxy cuticula of the leaf and subsequently diffuse, via the cuticula, into the tissue underneath, to the actual site of action.

It is generally known and agricultural practice to add certain adjuvants to formulations in order to improve the activity of the latter. Advantageously, this allows the amounts of active ingredient in the formulation to be reduced while maintaining the same activity, thereby being able to minimize costs and, optionally, operating within existing legislation. In individual cases, this also allows the spectrum of the active ingredient to be widened, since plants whose treatment with a specific active ingredient without addition was only possible to an unsatisfactory extent, are now capable of being subjected to such a treatment as the result of the addition of certain auxiliaries. Furthermore, the performance under adverse environmental conditions may be increased in individual cases by a suitable formulation. Of course, incompatibilities of various active ingredients in one formulation can also be avoided.

Such auxiliaries are sometimes also referred to adjuvants. Frequently, they take the form of surface-active or salt-like compounds. Depending on their mode of action, one can distinguish between, for example, modifiers, actuators, fertilizers and pH buffers. Modifiers influence the wetting, adhesion and spreading of a formulation. Actuators break the waxy plant cuticula and improve the penetration of the active ingredient into the cuticula, both in the short term (within minutes) and in the long term (within hours). Fertilizers such as ammonium sulfate, ammonium nitrate or urea improve the absorption and solubility of the active ingredient, and they may reduce antagonistic patterns of behavior of active ingredients. pH buffers are traditionally used for optimally adjusting the pH of the formulation.

As regards the uptake of the active ingredient into the leaf, surface-active substances may act as modifiers and actuators. In general, it is assumed that suitable surface-active substances are capable of increasing the effective contact area of fluids on leaves by reducing the surface tension. Moreover, certain surface-active substances are capable of dissolving or disrupting the epicuticular waxes, which facilitates the absorption of the active ingredient. Furthermore, some surface-active substances are also capable of improving the solubility of active ingredients in formulations, thereby avoiding, or at least delaying, crystal formation. Finally, in certain cases they can also influence the absorption of active ingredients by retaining moisture.

Adjuvants of the surface-active type are exploited in many ways for agrochemical purposes. They can be divided into anionic, cationic, nonionic or amphoteric groups of substances.

Petrol-based oils have traditionally been used as activating adjuvants. In recent times, seed extracts, natural oils and their derivatives, for example, from soybeans, sunflowers and coconut, have also been employed.

The synthetic surface-active substances which have usually been used as actuators take the form of, inter alia, polyoxyethylene condensates with alcohols, alkylphenols or alkylamines with HLB values in the range of from 8 to 13. In this regard, the document WO 00/42847 mentions for example the use of certain linear alcohol alkoxylates in order to increase the activity of agrochemical biocide formulations.

However, the spectrum of alcohol alkoxylates is varied. As surfactants, they are predominantly used in detergents and cleaners, in the metal-working industry, in the production and processing of textiles, in the leather industry, in papermaking, in the printing, electroplating and photographic industries, in water treatment, in pharmaceutical, veterinary and plant protection formulations, or in the plastics manufacturing and processing industries. It is in particular the structures of the alcohol moiety and in certain cases also those of the alkoxylate moiety which influence the properties of the alkoxylates so that a variety of technical effects come in useful in the abovementioned applications. These include wetting, spreading, penetration, adhesion, film formation, the improvement of compatibilities, drift control, and defoaming.

WO 03/090531 describes the use of alkoxylates of certain branched alcohols, which include in particular 2-propylheptanol, C13-oxo alcohols and C10-oxo alcohols, as adjuvant for the agrochemical sector. Similar alcohol alkoxylates are proposed in WO 2005/015998 specifically as adjuvant for fungicidal benzamide oxime derivatives. WO 00/35278 relates to agrochemical formulations based on PO/EO block copolymers of 2-ethylhexanol. The EO/PO-EO alkoxylates described in WO 99/03345 as adjuvant for improving the activity of a pesticide and plant protection composition are based on alcohols having more than 14 carbon atoms. WO 2005/084435 describes oil-based suspension concentrates which comprise one of the two end group-capped alcohol block alkoxylates CH₃—(CH₂)₁₀—O-(EO)—₆—(BO)₂—CH₃ or CH₃—(CH₂)₈—O-(EO)—₆—(BO)₂—CH₃ as penetrant. These end-group capped alcohol block alkoxylates are said to have a better activity than comparable alkoxylates without end group cap.

Thus, all of these alcohol alkoxylates are based on long-chain alcohols having at least 8 carbon atoms. However, individual alcohol alkoxylates based on short-chain alcohols are also known.

Thus, U.S. Pat. No. 6,071,857, for example, describes liquid pesticide compositions comprising the surface-active butanol EO/PO block alkoxylate known under the trade name Witconol NS 500 K®.

JP 02049701 describes crop protection granules comprising, as disintegrant, EO/PO alkoxylates of n-butanol.

EP 0 261 492 describes suspoemulsions comprising a surface-active ethylene oxide/propylene oxide block polymer having a relatively high average molecular weight for stabilizing the suspoemulsion.

JP 06313191 describes the use of the alcohol alkoxylates C₄H₉—O—(C₃H₆O)₂—(C₂H₄O)₈—(C₃H₆O)₂—H, C₄H₉—O—(C₃H₆O)₄—(C₂H₄O)₁₂—(C₃H₆O)₁—H and C₆H₁₃—O—(C₃H₆O)₂—(C₂H₄O)₁₂—(C₃H₆O)₁—H in detergents and cleaners.

GB 2 109 403 describes the use of a mono-C₁-C₆-alkyl ether of ethoxylate/propoxylate copolymers having an ethoxylate:propoxylate weight ratio in the range of from 30:70 to 5:95 in textile finishing compositions.

JP 2001 163813 describes alcohol alkoxylates of the C₄-C₈-alkyl-O—(C₂H₄O)₁₋₂—(C₃H₆O)_{0.1-1}—(C₂H₄O)₁₋₅—H type and their use as solvents or heat exchangers.

JP 09 031004 appears to describe EO/PO alkoxylates of heptanols and their use as solvents having surface-active and emulsifying action.

WO 2006/070816 appears to describe EO/PO alkoxylates of n-propanol and their use as surfactant for forming a stable foam.

U.S. Pat. No. 6,025,318 describes detergents and cleaners comprising EO/PO alkoxylates of iso-C₁₋₃-alcohols and hexanol.

However, none of these publications considers the use of the alcohol alkoxylates based on short-chain alcohols as activity-enhancing adjuvant in the crop protection field.

The present invention is based on the object of providing further adjuvants which are useful in the agrochemical sector.

This object is achieved by the present invention by means of specific alkoxylates of short-chain alcohols, including in particular PO/EO, EO/PO and EO/PO/EO block alkoxylates and BO/EO, EO/BO and EO/BO/EO block alkoxylates, their use as adjuvant, and agrochemical compositions which comprise these alkoxylates.

The present invention relates to alkoxylated alcohols of the formula (I)

R—O—(CₘH₂ₘO)_{q}—[(C₂H₄O)_{z}—(CₙH₂ₙO)ₓ—(C₂H₄O)_{y}]_{co}—Z (I)

in which
R represents C₁-C₇-alkyl;
m represents 2 or 3;
q represents 0, 1, 2 or 3;
n represents an integer from 3 to 16,
x represents a value of from 1 to 100;
y represents a value of from 0 to 100;
z represents a value of from 0 to 100;
x+y+z corresponds to a value of from 2 to 100; and
Z represents hydrogen or an end group cap,
where at least one of y and z is greater than 0.

According to one aspect, the following alcohol alkoxylates are per se (but not their use in the compositions and uses disclosed herein) excluded according to the invention:
a) C₄H₉—O—(C₃H₆O)₂—(C₂H₄O)₈—(C₃H₆O)₂—H
b) C₄H₉—O—(C₃H₆O)₄—(C₂H₄O)₁₂—(C₃H₆O)₁—H
c) C₆H₁₃—O—(C₃H₆O)₂—(C₂H₄O)₁₂—(C₃H₆O)₁—H.

According to a further aspect, the following alcohol alkoxylates are per se (but not their use in the compositions and uses disclosed herein) excluded according to the invention:
d) mono-C₁-C₆-alkyl ethers of ethoxylate/propoxylate copolymers having an ethoxylate:propoxylate weight ratio in the range from 30:70 to 5:95.

According to a further aspect, the following alcohol alkoxylates are per se (but not their use in the compositions and uses disclosed herein) excluded according to the invention:
e) C₄-C₈-alkyl-O—(C₂H₄O)₁₋₂—(C₃H₆O)_{0.1-1}—(C₂H₄O)₁₋₅—H.

According to a further aspect, the following alcohol alkoxylates are per se (but not their use in the compositions and uses disclosed herein) excluded according to the invention:
f) 1,2-bis-(2-methoxyethoxy)propane;
g) 1-ethoxy-2-(2-ethoxyethoxy)propane;
h) 1-O-(2-methoxyethyl)propane-1,2-diol; and
i) 4-methyl-2,5-dioxaheptan-7-ol.

Particular advantages result from the addition of the alcohol alkoxylates according to the invention to compositions which comprise active ingredients for the treatment of plants.

The present invention therefore also relates to compositions comprising

(a) at least one active ingredient for the treatment of plants; and
(b) at least one alkoxylated alcohol of the formula (I)

R—O—(CₘH₂ₘO)_{q}—[(C₂H₄O)_{z}—(CₙH₂ₙO)ₓ—(C₂H₄O)_{y}]_{co}—Z (I)

in which
R represents C₁-C₇-alkyl;
m represents 2 or 3;
q represents 0, 1, 2 or 3;
n represents an integer from 3 to 16;
x represents a value of from 1 to 100;
y represents a value of from 0 to 100;
z represents a value of from 0 to 100;
x+y+z corresponds to a value of from 2 to 100; and
Z represents hydrogen or an end group cap,
where at least one of y and z is greater than 0.

The alcohol alkoxylates which are present in the compositions according to the invention have in particular adjuvant, in particular activity-enhancing, properties. Thus, the addition of such alkoxylates makes possible a faster uptake of active ingredients by a plant to be treated with the active ingredient. The adjuvant activity gives rise in particular to the following aspects in the treatment of plants with one or more active ingredients:
a comparatively greater activity of the active ingredient at a given application rate;
a comparatively lower application rate at a given activity;
a comparatively greater uptake of the active ingredient by the plant, in particular via the leaf, and therefore advantages in the post-emergence method, in particular in the spray treatment of plants.

Accordingly, the present invention also relates to the use of an alkoxylated alcohol of the formula (I)

R—O—(CₘH₂ₘO)_{q}—[(C₂H₄O)_{z}—(CₙH₂ₙO)ₓ—(C₂H₄O)_{y}]_{co}—Z (I)

in which
R represents C₁-C₇-alkyl;
m represents 2 or 3;
q represents 0, 1, 2 or 3;
n represents an integer from 3 to 16;
x represents a value of from 1 to 100;
y represents a value of from 0 to 100;
z represents a value of from 0 to 100;
x+y+z corresponds to a value of from 2 to 100; and
Z represents hydrogen or an end group cap,
where at least one of y and z is greater than 0,
as adjuvant in the treatment of plants.

The use according to the invention is directed in particular to plant cultivation, to agriculture and to horticulture. In particular, it serves for controlling undesired plant growth.

Accordingly, the present invention also relates to methods corresponding to the above purposes for the treatment of plants, where a suitable amount of alkoxylate according to the invention is applied.

Special advantages are obtained in particular in the cultivation of Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica aleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Ribes sylvestre, Ricinus communis, Saccharum officinarum, Secale cereale, Sinapsis alba, Solanum tuberosum, Sorghum bicolor (s. vulgare), Theobroma cacao, Trifolium pratense, Triticale, Triticum aestivum, Triticum durum, Vicia faba, Vitis vinifera, Zea mays.

Special effects are obtained by the compositions according to the invention in the cultivation of Allium cepa, Hordeum vulgare, Triticum aestivum and Triticum durum.

Moreover, the alkoxylates to be used in accordance with the invention can also be used in crops which tolerate the action of pesticides, in particular of herbicides. Such crops can be obtained for example by breeding, but also by recombinant methods.

The alcohol moiety of the alcohol alkoxylates according to the invention is, as a rule, based on alcohols or alcohol mixtures having 1 to 7 and in particular either 1 to 3, 4, or 5 to 7, i.e. 5, 6 or 7, carbon atoms, which alcohols or alcohol mixtures are known per se. R in formula (I) thus represents in particular methyl, ethyl, n-propyl, isopropyl, n-butyl, 2-butyl, isobutyl or tert-butyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, 2-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 3-ethylbutyl, 1,1,2-trimethylpropyl, 1,2,2-trimethylpropyl, 1-ethyl-1-methylpropyl, 1-ethyl-2-methylpropyl, 2-ethyl-1-methylpropyl, 2-ethyl-2-methylpropyl, n-heptyl, 1-methylhexyl, 2-methylhexyl, 3-methylhexyl, 4-methylhexyl, 5-methylhexyl, 1,1-dimethylpentyl, 1,2-dimethylpentyl, 1,3-dimethylpentyl, 1,4-dimethylpentyl, 2,2-dimethylpentyl, 2,3-dimethylpentyl, 2,4-dimethylpentyl, 3,3-dimethylpentyl, 3,4-dimethylpentyl, 4,4-dimethylpentyl, 1-ethylpentyl, 2-ethylpentyl, 3-ethylpentyl, 4-ethylpentyl, 1,1,2-trimethylbutyl, 1,1,3-trimethylbutyl, 1,2,2-trimethylbutyl, 1,2,3-trimethylbutyl, 1,3,3-trimethylbutyl, 1-ethyl-1-methylbutyl, 1-ethyl-2-methylbutyl, 1-ethyl-3-methylbutyl, 2-ethyl-1-methylbutyl, 2-ethyl-2-methylbutyl, 2-ethyl-3-methylbutyl, 3-ethyl-1-methylbutyl, 3-ethyl-2-methylbutyl, 3-ethyl-3-methylbutyl, 1-propylbutyl, 2-propylbutyl, 3-propylbutyl, 1-butylpropyl, 2-butylpropyl, 1-propyl-1-methylpropyl, 1-propyl-2-methylpropyl, 2-propyl-1-methylpropyl, 2-propyl-2-methylpropyl, 1,1-diethylpropyl, 1,2-diethylpropyl or 2,1-diethylpropyl, it also being possible for mixtures of two or more alcohol alkoxylates in which R is different to be suitable.

The alcohol moiety of the alkoxylates is therefore linear or branched. The linear alcohols include in particular methanol, ethanol, n-propanol, n-butanol, n-pentanol and n-hexanol, moreover n-heptanol. Among the branched alcohols, the branched pentanols (amyl alcohols), which are frequently used as a mixture, must be mentioned in particular. Those having 5, 6 or 7 carbon atoms have proven to be particularly advantageous.

According to a particular embodiment, the alkoxylated alcohol is selected from among alkoxylated alcohols of the formula (I) where q is 1, 2 or 3. Such alcohol alkoxylates can be prepared in a targeted fashion, for example by alkoxylating in the above-described manner and, optionally, end-group capping, a compound of the formula (XI)

R—O—(CₘH₂ₘO)_{q}—H (XI)

in which
R, m and q are as defined herein.

The compounds of the formula (XI) are mono-C₁-C₇-alkyl ethers of ethylene glycol (m=2; q=1), propylene glycol (m=3; q=1), diethylene glycol (m=2; q=2), dipropylene glycol (m=3; q=2), triethylene glycol (m=2; q=3) or tripropylene glycol (m=3; q=3) which are known per se.

Of particular importance in accordance with the invention are the alkyl ethers of mono-, di- and tripropylene glycol of the formula (XIa)

R—O—(CH₂CH(CH₃)O)_{q}—H (XIa)

in which R and q are as defined herein.

In accordance with another aspect, the mono-C₁-C₄-alkyl ethers (i.e. R is in particular C₁-C₄-alkyl), and of these mainly the dipropylene glycol monoalkyl ethers (q=2), must be emphasized. These include, in particular, dipropylene glycol monomethyl ether, dipropylene glycol monoethyl ether, dipropylene glycol mono-n-propyl ether and dipropylene glycol mono-n-butyl ether.

Suitable alcohols can be obtained both from native sources and via the synthetic route, for example by composing them from starting materials with a lower number of carbon atoms.

The alkoxylation is the result of the reaction with suitable alkylene oxides, namely with ethylene oxide (EO) and at least one higher alkylene oxide having 3 to 16 and preferably 3 to 6 carbon atoms, of which in particular 1,2-propylene oxide (PO), 1,2-butylene oxide (BO), 1,2-pentylene oxide (PeO) and 1,2-hexylene oxide (HO) may be mentioned. Furthermore, 1,2-decylene oxide (DeO) may also be mentioned.

The respective degree of alkoxylation is the result of the charged amounts of alkylene oxide(s) and the reaction conditions selected for the reaction. The former is, as a rule, a statistical mean, since the number of alkylene oxide units of the alcohol alkoxylates which are the result of the reaction varies.

The degree of alkoxylation, i.e. the mean chain length of the polyether chains of alcohol alkoxylates according to the invention and their composition (in other words, the values of x, y, z) can be controlled by the ratio of the molar amounts of alcohol to ethylene oxide and higher alkylene oxide employed in their preparation, and by the reaction conditions. On the one hand, the alcohol alkoxylates according to the invention preferably comprise at least or more than approximately 2, more preferably at least or more than approximately 4, in particular at least or more than approximately 6, above all at least or more than approximately 8, and especially preferably at least or more than approximately 10 alkylene oxide units. On the other hand, the alcohol alkoxylates according to the invention preferably comprise not more than or less than approximately 100, 90 or 85, more preferably not more than or less than approximately 80, 70 or 65, in particular not more than or less than approximately 60, 50 or 45, above all not more than or less than approximately 40 or 35, and especially preferably not more than or less than approximately 30 or 25 alkylene oxide units. Accordingly, preference is given, for example, to alcohol alkoxylates having about 2 to 100, 90 or 85, such as about 4 to 80, 70 or 65, in particular about 6 to 60, 50 or 45, especially about 8 to 40 or 35, and particularly about 10 to 30 or 25 alkylene oxide units (x+y+z). If y=zero, preference is thus given, for example, to alcohol alkoxylates having about 2 to 100, 90 or 85, such as about 4 to 80, 70 or 65, in particular about 6 to 60, 50 or 45, especially about 8 to 40 or 35, and particularly about 10 to 30 or 25 alkylene oxide units (z+x). If z=zero, preference is thus given, for example, to alcohol alkoxylates having about 2 to 100, 90 or 85, such as about 4 to 80, 70 or 65, in particular about 6 to 60, 50 or 45, especially about 8 to 40 or 35, and particularly about 10 to 30 or 25 alkylene oxide units (x+y).

According to a particular embodiment, the invention relates to alcohol alkoxylates of the formula (I) in which the value of x is greater than the value of z, than the value of y or than the sum of z and y. These are alcohol alkoxylates having more units of the higher alkylene oxide than ethylene oxide units. This applies especially to alcohol alkoxylates of the formula (I), in which n is 3.

On the one hand, such alcohol alkoxylates have an alkoxylate moiety in which the ratio of higher alkylene oxide to ethylene oxide (x to z, x to y or x to (z+y)) is at least or more than 1.1:1, preferably at least or more than 1.5:1, especially at least or more than 1.8:1, and in particular at least or more than 2:1, for example at least or more than 3:1.

On the other hand, such alcohol alkoxylates have an alkoxylate moiety in which the ratio of higher alkylene oxide to ethylene oxide (x to z, x to y or x to (z+y)) is at most or less than 25:1, preferably at most or less than 15:1, and in particular at most or less than 10:1, for example at most or less than 4.3:1. Preference is thus given to alkoxylates in which the ratio of higher alkylene oxide to ethylene oxide (x to z, x to y or x to (z+y)) is from 1.1:1 to 25:1, preferably from 1.5:1 to 15:1, especially from 1.8 to 15:1, and in particular from 2:1 to 10:1, for example from 2.3:1 to 4.3:1. This applies especially to alcohol alkoxylates of the formula (I) in which n is 3 or 4.

According to a further particular embodiment, the invention relates to alcohol alkoxylates of the formula (I) in which the value x is less than the value of z, than the value of y or than the sum of z and y. These are alcohol alkoxylates having fewer units of the higher alkylene oxide than ethylene oxide units. This applies especially to alcohol alkoxylates of the formula (I) in which n is greater than 3, that is 4, or especially 5, 6 or 10.

On the one hand, such alcohol alkoxylates have an alkoxylate moiety in which the ratio of ethylene oxide to higher alkylene oxide (z to x, y to x or (z+y) to x) is at least or more than 1.1:1, preferably at least or more than 1.5:1, especially at least or more than 2:1, and in particular at least or more than 2.5:1, for example at least or more than 3:1. On the other hand, such alcohol alkoxylates have an alkoxylate moiety in which the ratio of ethylene oxide to higher alkylene oxide (z to x, y to x or (z+y) to x) is at most or less than 25:1, preferably at most or less than 20:1, and in particular at most or less than 15:1, for example at most or less than 10:1. Preference is thus given to alcohol alkoxylates in which the ratio of ethylene oxide to higher alkylene oxide (z to x, y to x or (z+y) to x) is from 1.1:1 to 25:1, preferably from 1.5:1 to 20:1 and in particular from 2:1 to 15:1, for example from 2.5:1 to 10:1. This applies especially to alcohol alkoxylates of the formula (I) in which n is greater than 3, i.e. especially 5, 6 or 10.

The degree of alkoxylation (value of x) which can be attributed to the higher alkylene oxide units is, on the one hand, generally from 1 to 50, preferably from 3 to 30 and in particular from 5 to 20. This applies especially to alcohol alkoxylates of the formula (I) in which n is 3 or 4.

The degree of alkoxylation (value of x) which can be attributed to the higher alkylene oxide units is, on the other hand, generally from 1 to 30, preferably from 2 to 20 and in particular from 3 to 15. This applies especially to alcohol alkoxylates of the formula (I) in which n is greater than 3, i.e. 4, or especially 5, 6 or 10.

The degree of alkoxylation (value of z, y or (z+y)) which can be attributed to the ethylene oxide units is, on the one hand, generally from 1 to 30, preferably from 2 to 20 and in particular from 3 to 10. This applies especially to alcohol alkoxylates of the formula (I) in which n is 3 or 4.

The degree of alkoxylation (value of z, y or (z+y)) which can be attributed to the ethylene oxide units is, on the other hand, generally from 1 to 50, preferably from 3 to 40 and in particular from 5 to 30. This applies especially to alcohol alkoxylates of the formula (I) in which n is greater than 3, i.e. 4, or especially 5, 6 or 10.

A particular embodiment are alcohol alkoxylates of the formula (I) in which z is zero, i.e. alkoxylated alcohols of the formula (Ia)

R—O—(CₘH₂ₘO)_{q}—[(CₙH₂ₙO)ₓ—(C₂H₄O)_{y}]_{co}—Z (Ia)

in which R, m, q, n, x, y, Z are as defined herein and y is greater than zero.

A further particular embodiment are alcohol alkoxylates of the formula (I) in which y and z are greater than zero.

The reaction of the alcohols or alcohol mixtures with the alkylene oxide(s) is carried out by customary processes known to the skilled worker and in apparatuses conventionally used for this purpose.

The alkoxylation can be catalyzed by strong bases, such as alkali metal hydroxides and alkaline earth metal hydroxides, Brønsted acids or Lewis acids such as AlCl₃, BF₃, and the like. Catalysts such as hydrotalcite or DMC may be used for alcohol alkoxylates with a narrow distribution.

The alkoxylation is preferably carried out at temperatures in the range of from approximately 80 to 250° C., preferably approximately 100 to 220° C. The pressure is preferably between ambient pressure and 600 bar. If desired, the alkylene oxide may comprise a mixture of inert gas, for example approximately 5 to 60%.

In the formulae (I) and (Ia), the alkylene oxide units and ethylene oxide units, if present, may be arranged in any way. Thus, the structural unit [ . . . ]_{co} can be a random copolymer, a gradient copolymer, an alternating copolymer or a block copolymer of alkylene oxide units CₙH₂ₙO and ethylene oxide units C₂H₄O.

—CₙH₂ₙO— (where n is 3 or greater) represents either —CH(Cₙ₋₂H₂ₙ₋₃)CH₂O— (for example —CH(CH₃)CH₂O—) or —CH₂CH(Cₙ₋₂H₂ₙ₋₃)O— (for example —CH₂CH(CH₃)O—). Here, a specific alcohol alkoxylate may comprise essentially alkylene oxide units of one or the other type, or both. An alkylene oxide block —(CₙH₂ₙO)ₓ— can be composed essentially of alkylene oxide units of the formula —CH₂CH(Cₙ₋₂H₂ₙ₋₃)O—, essentially of alkylene oxide units of the formula —CH(Cₙ₋₂H₂ₙ₋₃)CH₂O—, or both of alkylene oxide units of the formula —CH₂CH(Cₙ₋₂H₂ₙ₋₃)O— and of alkylene oxide units of the formula —CH(Cₙ₋₂H₂ₙ₋₃)CH₂O—, where, in the latter case, the two alkylene oxide units can be randomly distributed, alternating or arranged in two or more sub-blocks. The base-catalyzed alkoxylation generates predominantly alkylene oxide units of the formula —CH₂CH(Cₙ₋₂H₂ₙ₋₃)O—, since the attack of the anion preferably takes place at the sterically less hindered secondary carbon atom of the alkylene oxide. Customary molar ratios are more than 60:40, 70:30 or 80:20, for example approximately 85:15, in favor of alkylene oxide units of the formula —CH₂CH(Cₙ₋₂H₂ₙ₋₃)O—.

According to a particular embodiment, the alkoxylated alcohol is selected from alcohol block alkoxylates of the formula (II)

R—O—(CₘH₂ₘO)_{q}—(C₂H₄O)_{z}—(CₙH₂ₙO)ₓ—(C₂H₄O)_{y}—Z (II)

in which R, m, q, n, x, y, z, Z are as defined herein. If both z and y are greater than zero, these are triblock alkoxylates, for example EO/PO/EO block alkoxylates.

The alcohol block alkoxylates of the formula (II) include in particular alkoxylated alcohols of the formula (IIa)

R—O—(CₘH₂ₘO)_{q}—(CₙH₂ₙO)ₓ—(C₂H₄O)_{y}—Z (IIa),

in which, m, q, n, x, y, Z are as defined herein and y is greater than zero. These are alcohol alkoxylates having an ethylene oxide block and a higher alkylene oxide block, where the ethylene oxide block is arranged terminally, for example PO/EO, BO/EO, PeO/EO or DeO/PO block alkoxylates.

The alcohol block alkoxylates of the formula (II) also include in particular alkoxylated alcohols of the formula (IIb)

R—O—(CₘH₂ₘO)_{q}—(C₂H₄O)_{z}—(CₙH₂ₙO)ₓ—Z (IIb),

in which R, m, q, n, x, z, Z are as defined herein and z is greater than zero. These are alcohol alkoxylates having an ethylene oxide block and a higher alkylene oxide block, where the higher alkylene oxide block is arranged terminally, for example EO/PO, EO/BO, EO/PeO or EO/DeO block alkoxylates.

From among the alkoxylated alcohols described herein, particular preference is given to those in which n=3, i.e. in which the higher alkylene oxide unit is a propylene oxide unit. These include especially alcohol block alkoxylates of the formula (III)

R—O—(CₘH₂ₘO)_{q}—(C₃H₆O)ₓ—(C₂H₄O)_{y}—Z (III)

in which
R is C₁-C₇-alkyl;
m is 2 or 3;
q is 0, 1, 2, or 3;
x has a value from 1 to 100;
y has a value from 1 to 100;
x+y corresponds to a value of from 2 to 100; and
Z is hydrogen or an end group cap.

This type of alcohol alkoxylate according to the invention is based on at least one propylene oxide block and at least one ethylene oxide block, with the ethylene oxide block being in the terminal position. Further particular embodiments result from what has been said in connection with the alcohol alkoxylates of the formulae (I), (Ia), (II) and (IIa).

In a further particular aspect of the invention, the alcohol alkoxylates of the formula (III) according to the invention have at least approximately 5 or more, preferably at least approximately 8 or more, in particular at least approximately 12 or more propylene oxide units (value of x). On the other hand, the alcohol alkoxylates of the formula (III) according to the invention have, in a further particular aspect of the invention, not more than approximately 35 or less, preferably not more than approximately 25 or less and in particular not more than approximately 20 or less propylene oxide units (value of x). Accordingly, preferred alcohol alkoxylates of the formula (IV) are those which comprise approximately 5 to 35, preferably approximately 8 to 25 and in particular approximately 12 to 20 propylene oxide units (value of x).

In a further particular aspect of the invention, the alcohol alkoxylates of the formula (III) according to the invention have at least approximately 2 or more, preferably at least approximately 3 or more, in particular at least approximately 5 or more ethylene oxide units (value of y). On the other hand, the alcohol alkoxylates of the formula (III) according to the invention have, in a further particular aspect of the invention, not more than approximately 30 or less, preferably not more than approximately 20 or less and in particular not more than approximately 12 or less ethylene oxide units (value of y). Accordingly, preferred alcohol alkoxylates of the formula (IV) are those which comprise approximately 2 to 30, preferably approximately 3 to 20 and in particular approximately 5 to 12 ethylene oxide units (value of y).

In a further particular aspect of the invention, the alcohol alkoxylates of the formula (III) according to the invention have an alkoxylate moiety in which the ratio of ethylene oxide to propylene oxide (y to x) is at least 1:5 or more, preferably at least 1:4 or more, and in particular at least 1:3 or more. On the other hand, the alcohol alkoxylates of the formula (III) according to the invention have, in a further particular aspect of the invention, an alkoxylate moiety in which the ratio of ethylene oxide to propylene oxide (y to x) is not more than 2:1 or less, preferably not more than 3:2 or less, and in particular not more than 1:1 or less. Accordingly, preferred alkoxylates of the formula (III) are those in which the ratio of ethylene oxide to propylene oxide (y to x) is 1:5 to 2:1, preferably 1:4 to 3:2 and in particular 1:3 to 1:1.

From among the alkoxylated alcohols described herein, particular preference is furthermore given to those in which n=4, i.e. in which the higher alkylene oxide unit is a butylene oxide unit. These include especially alcohol block alkoxylates of the formula (IV)

R—O—(CₘH₂ₘO)_{q}—(C₄H₈O)ₓ—(C₂H₄O)_{y}—Z (IV)

in which
R is C₁-C₇-alkyl;
m is 2 or 3;
q is 0, 1, 2, or 3;
x has a value from 1 to 100;
y has a value from 1 to 100;
x+y corresponds to a value of from 2 to 100; and
Z is hydrogen or an end group cap.

This type of alcohol alkoxylate according to the invention is based on a butylene oxide block and at least one ethylene oxide block, with the ethylene oxide block being in the terminal position. Further particular embodiments result from what has been said in connection with the alcohol alkoxylates of the formulae (I), (Ia), (II) and (IIa).

The alcohol alkoxylates of the formula (IV) according to the invention have a hydrophobic moiety (R—O—(CₘH₂ₘO)_{q}—(C₄H₈O)ₓ—) and a hydrophilic moiety (—(C₂H₄O)_{y}—Z). If q is greater than zero, then m is preferably 3. A particular hydrophobic moiety is produced if q is zero (R—O—(C₄H₈O)ₓ—).

In accordance with a particular embodiment, the hydrophobic moiety of the alcohol alkoxylates of the formula (IV) according to the invention comprises at least 15 carbon atoms in the radical R and the group —(C₄H₈O)ₓ— (in other words, the total of (4·x) and the number of the carbon atoms in R is at least 15). Preferably, the hydrophobic moiety of the alcohol alkoxylates according to the invention has at least 17 and in particular at least 19 carbon atoms. On the other hand, the hydrophobic moiety of the alcohol alkoxylates has in accordance with a further aspect of the invention not more than 60, preferably not more than 55 or 50 and in particular not more than 46 carbon atoms.

In accordance with a further particular embodiment, the hydrophobic moiety of the alcohol alkoxylates of the formula (IV) according to the invention comprises at least 2 branches. Here, at least 1 branch is located in the alkoxylate moiety. On the other hand, the hydrophobic moiety of the alcohol alkoxylates comprises, in a further aspect of the invention, not more than 12, preferably not more than 11 or 10, and in particular not more than 9 branches. In fact, each butylene oxide contributes one branch, which is why the number of branches in the hydrophobic alkoxylate moiety corresponds to the total of the carbon atoms contributed by the butylene oxide units, i.e. (4·x).

In accordance with a further particular embodiment, the hydrophobic moiety of the alcohol alkoxylates of the formula (IV) according to the invention comprises at least approximately 0.1, preferably at least approximately 0.13 and in particular at least approximately 0.16 branches per C atom. On the other hand, the hydrophobic moiety of the alcohol alkoxylates comprises, in a further aspect of the invention, not more than 0.3, preferably not more than 0.27 and in particular not more than 0.25 branches.

In a further particular aspect of the invention, the alcohol alkoxylates of the formula (IV) according to the invention have at least approximately 2 or more, preferably at least approximately 3 or more, in particular at least approximately 4.5 or more butylene oxide units (value of x). On the other hand, the alcohol alkoxylates of the formula (IV) according to the invention have, in a further particular aspect of the invention, not more than approximately 15 or less, preferably not more than approximately 12 or less and in particular not more than approximately 9 or less butylene oxide units (value of x). Accordingly, preferred alcohol alkoxylates of the formula (IV) are those which comprise approximately 2 to 15, preferably approximately 3 to 12 and in particular approximately 4.5 to 9 butylene oxide units (value of x).

In a further particular aspect of the invention, the alcohol alkoxylates of the formula (IV) according to the invention have at least approximately 2 or more, preferably at least approximately 3 or more, in particular at least approximately 5 or more ethylene oxide units (value of y). On the other hand, the alcohol alkoxylates of the formula (IV) according to the invention have, in a further particular aspect of the invention, not more than approximately 30 or less, preferably not more than approximately 20 or less and in particular not more than approximately 12 or less ethylene oxide units (value of y). Accordingly, preferred alcohol alkoxylates of the formula (IV) are those which comprise approximately 2 to 30, preferably approximately 3 to 20 and in particular approximately 5 to 12 ethylene oxide units (value of y).

In a further particular aspect of the invention, the alcohol alkoxylates of the formula (IV) according to the invention have an alkoxylate moiety in which the ratio of ethylene oxide to butylene oxide (y to x) is at least 1:4 or more, preferably at least 1:3 or more, and in particular at least 1:2 or more. On the other hand, the alcohol alkoxylates of the formula (IV) according to the invention have, in a further particular aspect of the invention, an alkoxylate moiety in which the ratio of ethylene oxide to butylene oxide (y to x) is not more than 8:1 or less, preferably not more than 5:1 or less, and in particular not more than 3:1 or less. Accordingly, preferred alkoxylates of the formula (IV) are those in which the ratio of ethylene oxide to butylene oxide (y to x) is 1:4 to 8:1, preferably 1:3 to 5:1 and in particular 1:2 to 3:1.

From among the alkoxylated alcohols described herein, particular preference is furthermore given to those in which n=5, i.e. in which the higher alkylene oxide unit is a pentylene oxide unit. These include especially alcohol block alkoxylates of the formula (V)

R—O—(CₘH₂ₘO)_{q}—(C₅H₁₀O)ₓ—(C₂H₄O)_{y}—Z (V)

in which
R is C₁-C₇-alkyl;
m is 2 or 3;
q is 0, 1, 2, or 3;
x has a value from 1 to 100;
y has a value from 1 to 100;
x+y corresponds to a value of from 2 to 100; and
Z is hydrogen or an end group cap.

This type of alcohol alkoxylate according to the invention is based on a pentylene oxide block and at least one ethylene oxide block, with the ethylene oxide block being in the terminal position. Further particular embodiments result from what has been said in connection with the alcohol alkoxylates of the formulae (I), (Ia), (II) and (IIa).

The alcohol alkoxylates of the formula (V) according to the invention also have a hydrophobic moiety (R—O—(CₘH₂ₘO)_{q}—(C₅H₁₀O)ₓ—) and a hydrophilic moiety (—(C₂H₄O)_{y}—Z). If q is greater than zero, then m is preferably 3. A particular hydrophobic moiety is produced if q is zero (R—O—(C₅H₁₀O)ₓ—).

In accordance with a particular embodiment, the hydrophobic moiety of the alcohol alkoxylates of the formula (V) according to the invention comprises at least 15 carbon atoms in the radical R and in the group —(C₅H₁₀O)ₓ— (in other words, the total of (5·x) and the number of the carbon atoms in R is at least 15). Preferably, the hydrophobic moiety of the alcohol alkoxylates according to the invention has at least 17 and in particular at least 19 carbon atoms. On the other hand, the hydrophobic moiety of the alcohol alkoxylates has, in a further aspect of the invention, not more than 60, preferably not more than 55 or 50 and in particular not more than 46 carbon atoms.

In accordance with a further particular embodiment, the hydrophobic moiety of the alcohol alkoxylates of the formula (V) according to the invention comprises at least 2 branches. Here, at least 1 branch is located in the alkoxylate moiety. On the other hand, the hydrophobic moiety of the alcohol alkoxylates comprises, in a further aspect of the invention, not more than 12, preferably not more than 11 or 10, and in particular not more than 9 branches. In fact, each pentylene oxide contributes one branch, which is why the number of branches in the hydrophobic alkoxylate moiety corresponds to the total of the carbon atoms contributed by the pentylene oxide units, i.e. (5·x).

In accordance with a further particular embodiment, the hydrophobic moiety of the alcohol alkoxylates of the formula (V) according to the invention comprises at least approximately 0.1, preferably at least approximately 0.13 and in particular at least approximately 0.16 branches per C atom. On the other hand, the hydrophobic moiety of the alcohol alkoxylates comprises, in a further aspect of the invention, not more than 0.3, preferably not more than 0.27 and in particular not more than 0.25 branches.

In a further particular aspect of the invention, the alcohol alkoxylates of the formula (V) according to the invention have at least approximately 2 or more, preferably at least approximately 3 or more, in particular at least approximately 4 or more pentylene oxide units (value of x). On the other hand, the alcohol alkoxylates of the formula (V) according to the invention have, in a further particular aspect of the invention, not more than approximately 15 or less, preferably not more than approximately 12 or less and in particular not more than approximately 9 or less pentylene oxide units (value of x). Accordingly, preferred alcohol alkoxylates of the formula (V) are those which comprise approximately 2 to 15, preferably approximately 3 to 12 and in particular approximately 4 to 9 pentylene oxide units (value of x).

In a further particular aspect of the invention, the alcohol alkoxylates of the formula (V) according to the invention have at least approximately 2 or more, preferably at least approximately 3 or more, in particular at least approximately 5 or more ethylene oxide units (value of y). On the other hand, the alcohol alkoxylates of the formula (V) according to the invention have, in a further particular aspect of the invention, not more than approximately 30 or less, preferably not more than approximately 20 or less and in particular not more than approximately 12 or less ethylene oxide units (value of y). Accordingly, preferred alcohol alkoxylates of the formula (V) are those which comprise approximately 2 to 30, preferably approximately 3 to 20 and in particular approximately 5 to 12 ethylene oxide units (value of y).

In a further particular aspect of the invention, the alcohol alkoxylates of the formula (V) according to the invention have an alkoxylate moiety in which the ratio of ethylene oxide to pentylene oxide (y to x) is at least 1:4 or more, preferably at least 1:3 or more, and in particular at least 1:2 or more. On the other hand, the alcohol alkoxylates of the formula (V) according to the invention have, in a further particular aspect of the invention, an alkoxylate moiety in which the ratio of ethylene oxide to pentylene oxide (y to x) is not more than 8:1 or less, preferably not more than 5:1 or less, and in particular not more than 3:1 or less. Accordingly, preferred alkoxylates of the formula (V) are those in which the ratio of ethylene oxide to pentylene oxide (y to x) is 1:4 to 8:1, preferably 1:3 to 3:1 and in particular 1:2 to 5:1.

From among the alkoxylated alcohols described herein, particular preference is furthermore given to those in which n=10, i.e. in which the higher alkylene oxide unit is a decylene oxide unit. These include especially alcohol block alkoxylates of the formula (VI)

R—O—(CₘH₂ₘO)_{q}—(C₁₀H₂₀O)ₓ—(C₂H₄O)_{y}—Z (VI)

in which
R is C₁-C₇-alkyl;
m is 2 or 3;
q is 0, 1, 2, or 3;
x has a value from 1 to 100;
y has a value from 1 to 100;
x+y corresponds to a value of from 2 to 100; and
Z is hydrogen or an end group cap.

This type of alcohol alkoxylate according to the invention is based on a decylene oxide block and at least one ethylene oxide block, with the ethylene oxide block being in the terminal position. Further particular embodiments result from what has been said in connection with the alcohol alkoxylates of the formulae (I), (Ia), (II) and (IIa).

The alcohol alkoxylates of the formula (VI) according to the invention also have a hydrophobic moiety (R—O—(CₘH₂ₘO)_{q}—(C₁₀H₂₀O)ₓ—) and a hydrophilic moiety (—(C₂H₄O)_{y}—Z). If q is greater than zero, then m is preferably 3. A particular hydrophobic moiety is produced if q is zero (R—O—(C₁₀H₂₀O)ₓ—).

In accordance with a particular embodiment, the hydrophobic moiety of the alcohol alkoxylates of the formula (VI) according to the invention comprises at least 15 carbon atoms in the radical R and in the group —(C₁₀H₂₀O)ₓ— (in other words, the total of (10 ·x) and the number of the carbon atoms in R is at least 15). Preferably, the hydrophobic moiety of the alcohol alkoxylates according to the invention has at least 17 and in particular at least 19 carbon atoms. On the other hand, the hydrophobic moiety of the alcohol alkoxylates, has in a further aspect of the invention, not more than 60, preferably not more than 55 or 50 and in particular not more than 46 carbon atoms.

In accordance with a further particular embodiment, the hydrophobic moiety of the alcohol alkoxylates of the formula (VI) according to the invention comprises at least 2 branches. Here, at least 1 branch is located in the alkoxylate moiety. On the other hand, the hydrophobic moiety of the alcohol alkoxylates comprises, in a further aspect of the invention, not more than 12, preferably not more than 11 or 10, and in particular not more than 9 branches. In fact, each decylene oxide contributes one branch, which is why the number of branches in the hydrophobic alkoxylate moiety corresponds to the total of the carbon atoms contributed by the decylene oxide units, i.e. (10 ·x).

In accordance with a further particular embodiment, the hydrophobic moiety of the alcohol alkoxylates of the formula (VI) according to the invention comprises at least approximately 0.1, preferably at least approximately 0.13 and in particular at least approximately 0.16 branches per C atom. On the other hand, the hydrophobic moiety of the alcohol alkoxylates comprises, in a further aspect of the invention, not more than 0.3, preferably not more than 0.27 and in particular not more than 0.25 branches.

In a further particular aspect of the invention, the alcohol alkoxylates of the formula (VI) according to the invention have at least approximately 1 or more, preferably at least approximately 1.5 or more, in particular at least approximately 2 or more decylene oxide units (value of x). On the other hand, the alcohol alkoxylates of the formula (VI) according to the invention have, in a further particular aspect of the invention, not more than approximately 5 or less, preferably not more than approximately 4 or less and in particular not more than approximately 3 or less decylene oxide units (value of x). Accordingly, preferred alcohol alkoxylates of the formula (VI) are those which comprise approximately 1 to 5, preferably approximately 1.5 to 4 and in particular approximately 2 to 3 decylene oxide units (value of x).

In a further particular aspect of the invention, the alcohol alkoxylates of the formula (VI) according to the invention have at least approximately 2 or more, preferably at least approximately 3 or more, in particular at least approximately 5 or more ethylene oxide units (value of y). On the other hand, the alcohol alkoxylates of the formula (VI) according to the invention have, in a further particular aspect of the invention, not more than approximately 30 or less, preferably not more than approximately 20 or less and in particular not more than approximately 12 or less ethylene oxide units (value of y). Accordingly, preferred alcohol alkoxylates of the formula (V) are those which comprise approximately 2 to 30, preferably approximately 3 to 20 and in particular approximately 5 to 12 ethylene oxide units (value of y).

In a further particular aspect of the invention, the alcohol alkoxylates of the formula (VI) according to the invention have an alkoxylate moiety in which the ratio of ethylene oxide to decylene oxide (y to x) is at least 1:1 or more, preferably at least 3:1 or more, and in particular at least 5:1 or more. On the other hand, the alcohol alkoxylates of the formula (VI) according to the invention have, in accordance with a further particular aspect of the invention, an alkoxylate moiety in which the ratio of ethylene oxide to decylene oxide (y to x) is not more than 30:1 or less, preferably not more than 15:1 or less, and in particular not more than 10:1 or less. Accordingly, preferred alkoxylates of the formula (VI) are those in which the ratio of ethylene oxide to decylene oxide (y to x) is 1:1 to 30:1, preferably 3:1 to 15:1 and in particular 5:1 to 10:1.

The alkoxylated alcohols described herein in which n=3 also include alcohol block alkoxylates of the formula (VII)

R—O—(CₘH₂ₘO)_{q}—(C₂H₄O)_{z}—(C₃H₆O)ₓ—Z (VII)

in which
R is C₁-C₇-alkyl;
m is 2 or 3;
q is 0, 1, 2, or 3;
x has a value from 1 to 100;
z has a value from 1 to 100;
x+z corresponds to a value of from 2 to 100; and
Z is hydrogen or an end group cap.

This type of alcohol alkoxylate according to the invention is based on at least one propylene oxide block and at least one ethylene oxide block, with the propylene oxide block being in the terminal position. Further particular embodiments result from what has been said in connection with the alcohol alkoxylates of the formulae (I), (Ia), (II) and (IIb).

In a further particular aspect of the invention, the alcohol alkoxylates of the formula (VII) according to the invention have at least approximately 5 or more, preferably at least approximately 8 or more, in particular at least approximately 12 or more propylene oxide units (value of x). On the other hand, the alcohol alkoxylates of the formula (VII) according to the invention have, in a further particular aspect of the invention, not more than approximately 35 or less, preferably not more than approximately 25 or less and in particular not more than approximately 20 or less propylene oxide units (value of x). Accordingly, preferred alcohol alkoxylates of the formula (VII) are those which comprise approximately 5 to 35, preferably approximately 8 to 25 and in particular approximately 12 to 20 propylene oxide units (value of x).

In a further particular aspect of the invention, the alcohol alkoxylates of the formula (VII) according to the invention have at least approximately 2 or more, preferably at least approximately 3 or more, in particular at least approximately 5 or more ethylene oxide units (value of z). On the other hand, the alcohol alkoxylates of the formula (VII) according to the invention have, in a further particular aspect of the invention, not more than approximately 30 or less, preferably not more than approximately 20 or less and in particular not more than approximately 12 or less ethylene oxide units (value of z). Accordingly, preferred alcohol alkoxylates of the formula (VII) are those which comprise approximately 2 to 30, preferably approximately 3 to 20 and in particular approximately 5 to 12 ethylene oxide units (value of z).

In a further particular aspect of the invention, the alcohol alkoxylates of the formula (VII) according to the invention have an alkoxylate moiety in which the ratio of ethylene oxide to propylene oxide (z to x) is at least 1:5 or more, preferably at least 1:4 or more, and in particular at least 1:3 or more. On the other hand, the alcohol alkoxylates of the formula (VII) according to the invention have, in a further particular aspect of the invention, an alkoxylate moiety in which the ratio of ethylene oxide to propylene oxide (z to x) is not more than 2:1 or less, preferably not more than 3:2 or less, and in particular not more than 1:1 or less. Accordingly, preferred alkoxylates of the formula (VII) are those in which the ratio of ethylene oxide to propylene oxide (z to x) is 1:5 to 2:1, preferably 1:4 to 3:2 and in particular 1:3 to 1:1.

The alkoxylated alcohols described herein in which n=4 also include alcohol block alkoxylates of the formula (VIII)

R—O—(CₘH₂ₘO)_{q}—(C₂H₄O)_{z}—(C₄H₈O)ₓ—Z (VIII)

in which
R is C₁-C₇-alkyl;
m is 2 or 3;
q is 0, 1, 2, or 3;
x has a value from 1 to 100;
zy has a value from 1 to 100;
x+z corresponds to a value of from 2 to 100; and
Z is hydrogen or an end group cap.

This type of alcohol alkoxylate according to the invention is based on a butylene oxide block and at least one ethylene oxide block, with the butylene oxide block being in the terminal position. Further particular embodiments result from what has been said in connection with the alcohol alkoxylates of the formulae (I), (Ia), (II) and (IIb).

The alcohol alkoxylates of the formula (VIII) according to the invention have a hydrophobic moiety (—(C₄H₈O)ₓ—Z) and a hydrophilic moiety (—CₘH₂ₘO)_{q}—(C₂H₄O)_{z}).

In accordance with a particular embodiment, the hydrophobic moiety of the alcohol alkoxylates of the formula (VIII) according to the invention comprises at least 15 carbon atoms in the radical Z and in the group —(C₄H₈O)ₓ— (in other words, the total of (4·x) and the number of the carbon atoms in Z is at least 15). Preferably, the hydrophobic moiety of the alcohol alkoxylates according to the invention has at least 17 and in particular at least 19 carbon atoms. On the other hand, the hydrophobic moiety of the alcohol alkoxylates has, in a further aspect of the invention, not more than 60, preferably not more than 55 or 50 and in particular not more than 46 carbon atoms.

In accordance with a further particular embodiment, the hydrophobic moiety of the alcohol alkoxylates of the formula (VIII) according to the invention comprises at least 2 branches. Here, at least 1 branch is located in the alkoxylate moiety. On the other hand, the hydrophobic moiety of the alcohol alkoxylates comprises, in a further aspect of the invention, not more than 12, preferably not more than 11 or 10, and in particular not more than 9 branches. In fact, each butylene oxide contributes one branch, which is why the number of branches in the hydrophobic alkoxylate moiety corresponds to the total of the carbon atoms contributed by the butylene oxide units, i.e. (4·x).

In accordance with a further particular embodiment, the hydrophobic moiety of the alcohol alkoxylates of the formula (VIII) according to the invention comprises at least approximately 0.1, preferably at least approximately 0.13 and in particular at least approximately 0.16 branches per C atom. On the other hand, the hydrophobic moiety of the alcohol alkoxylates comprises, in a further aspect of the invention, not more than 0.3, preferably not more than 0.27 and in particular not more than 0.25 branches.

In a further particular aspect of the invention, the alcohol alkoxylates of the formula (VIII) according to the invention have at least approximately 2 or more, preferably at least approximately 3 or more, in particular at least approximately 4.5 or more butylene oxide units (value of x). On the other hand, the alcohol alkoxylates of the formula (VIII) according to the invention have, in a further particular aspect of the invention, not more than approximately 15 or less, preferably not more than approximately 12 or less and in particular not more than approximately 9 or less butylene oxide units (value of x). Accordingly, preferred alcohol alkoxylates of the formula (VIII) are those which comprise approximately 2 to 15, preferably approximately 3 to 12 and in particular approximately 4.5 to 9 butylene oxide units (value of x).

In a further particular aspect of the invention, the alcohol alkoxylates of the formula (VIII) according to the invention have at least approximately 2 or more, preferably at least approximately 3 or more, in particular at least approximately 5 or more ethylene oxide units (value of z). On the other hand, the alcohol alkoxylates of the formula (VIII) according to the invention have, in a further particular aspect of the invention, not more than approximately 30 or less, preferably not more than approximately 20 or less and in particular not more than approximately 12 or less ethylene oxide units (value of z). Accordingly, preferred alcohol alkoxylates of the formula (VIII) are those which comprise approximately 2 to 30, preferably approximately 2 to 20 and in particular approximately 5 to 12 ethylene oxide units (value of z).

In a further particular aspect of the invention, the alcohol alkoxylates of the formula (VIII) according to the invention have an alkoxylate moiety in which the ratio of ethylene oxide to butylene oxide (z to x) is at least 1:4 or more, preferably at least 1:3 or more, and in particular at least 1:2 or more. On the other hand, the alcohol alkoxylates of the formula (VIII) according to the invention have, in a further particular aspect of the invention, an alkoxylate moiety in which the ratio of ethylene oxide to butylene oxide (z to x) is not more than 8:1 or less, preferably not more than 5:1 or less, and in particular not more than 3:1 or less. Accordingly, preferred alkoxylates of the formula (VIII) are those in which the ratio of ethylene oxide to butylene oxide (z to x) is 1:4 to 8:1, preferably 1:3 to 5:1 and in particular 1:2 to 3:1.

The alkoxylated alcohols described herein in which n=5 also include alcohol block alkoxylates of the formula (IX)

R—O—(CₘH₂ₘO)_{q}—(C₂H₄O)_{z}—(C₅H₁₀O)ₓ—Z (IX)

in which
R is C₁-C_{T}-alkyl;
m is 2 or 3;
q is 0, 1, 2, or 3;
x has a value from 1 to 100;
z has a value from 1 to 100;
x+z corresponds to a value of from 2 to 100; and
Z is hydrogen or an end group cap.

This type of alcohol alkoxylate according to the invention is based on a pentylene oxide block and at least one ethylene oxide block, with the pentylene oxide block being in the terminal position. Further particular embodiments result from what has been said in connection with the alcohol alkoxylates of the formulae (I), (Ia), (II) and (IIa).

The alcohol alkoxylates of the formula (IX) according to the invention also have a hydrophobic moiety (—(C₅H₁₀O)ₓ—Z) and a hydrophilic moiety (—(CₘH₂ₘO)_{q}—(C₂H₄O)_{z}—).

In accordance with a particular embodiment, the hydrophobic moiety of the alcohol alkoxylates of the formula (IX) according to the invention comprises at least 15 carbon atoms in the radical R and in the group —(C₅H₁₀O)ₓ— (in other words, the total of (5·x) and the number of the carbon atoms in Z is at least 15). Preferably, the hydrophobic moiety of the alcohol alkoxylates according to the invention has at least 17 and in particular at least 19 carbon atoms. On the other hand, the hydrophobic moiety of the alcohol alkoxylates has, in a further aspect of the invention, not more than 60, preferably not more than 55 or 50 and in particular not more than 46 carbon atoms.

In accordance with a further particular embodiment, the hydrophobic moiety of the alcohol alkoxylates of the formula (IX) according to the invention comprises at least 2 branches. Here, at least 1 branch is located in the alkoxylate moiety. On the other hand, the hydrophobic moiety of the alcohol alkoxylates comprises, in a further aspect of the invention, not more than 12, preferably not more than 11 or 10, and in particular not more than 9 branches. In fact, each pentylene oxide contributes one branch, which is why the number of branches in the hydrophobic alkoxylate moiety corresponds to the total of the carbon atoms contributed by the pentylene oxide units, i.e. (5·x).

In accordance with a further particular embodiment, the hydrophobic moiety of the alcohol alkoxylates of the formula (IX) according to the invention comprises at least approximately 0.1, preferably at least approximately 0.13 and in particular at least approximately 0.16 branches per C atom. On the other hand, the hydrophobic moiety of the alcohol alkoxylates comprises, in a further aspect of the invention, not more than 0.3, preferably not more than 0.27 and in particular not more than 0.25 branches.

In a further particular aspect of the invention, the alcohol alkoxylates of the formula (IX) according to the invention have at least approximately 2 or more, preferably at least approximately 3 or more, in particular at least approximately 4 or more pentylene oxide units (value of x). On the other hand, the alcohol alkoxylates of the formula (IX) according to the invention have, in a further particular aspect of the invention, not more than approximately 15 or less, preferably not more than approximately 12 or less and in particular not more than approximately 9 or less pentylene oxide units (value of x). Accordingly, preferred alcohol alkoxylates of the formula (IX) are those which comprise approximately 2 to 15, preferably approximately 3 to 12 and in particular approximately 4 to 9 pentylene oxide units (value of x).

In a further particular aspect of the invention, the alcohol alkoxylates of the formula (IX) according to the invention have at least approximately 2 or more, preferably at least approximately 3 or more, in particular at least approximately 5 or more ethylene oxide units (value of z). On the other hand, the alcohol alkoxylates of the formula (IX) according to the invention have, in a further particular aspect of the invention, not more than approximately 30 or less, preferably not more than approximately 20 or less and in particular not more than approximately 12 or less ethylene oxide units (value of z). Accordingly, preferred alcohol alkoxylates of the formula (IX) are those which comprise approximately 2 to 30, preferably approximately 3 to 20 and in particular approximately 5 to 12 ethylene oxide units (value of z).

In a further particular aspect of the invention, the alcohol alkoxylates of the formula (IX) according to the invention have an alkoxylate moiety in which the ratio of ethylene oxide to pentylene oxide (z to x) is at least 1:4 or more, preferably at least 1:3 or more, and in particular at least 1:2 or more. On the other hand, the alcohol alkoxylates of the formula (IX) according to the invention have, in a further particular aspect of the invention, an alkoxylate moiety in which the ratio of ethylene oxide to pentylene oxide (z to x) is not more than 8:1 or less, preferably not more than 5:1 or less, and in particular not more than 3:1 or less. Accordingly, preferred alkoxylates of the formula (IX) are those in which the ratio of ethylene oxide to pentylene oxide (z to x) is 1:4 to 8:1, preferably 1:3 to 5:1 and in particular 1:2 to 3:1.

The alkoxylated alcohols described herein in which n=10 also include alcohol block alkoxylates of the formula (X)

R—O—(CₘH₂ₘO)_{q}—(C₂H₄O)_{z}(C₁₀H₂₀O)ₓ—Z (X)

in which
R is C₁-C₇-alkyl;
m is 2 or 3;
q is 0, 1, 2, or 3;
x has a value from 1 to 100;
z has a value from 1 to 100;
x+z corresponds to a value of from 2 to 100; and
Z is hydrogen or an end group cap.

This type of alcohol alkoxylate according to the invention is based on a decylene oxide block and at least one ethylene oxide block, with the decylene oxide block being in the terminal position. Further particular embodiments result from what has been said in connection with the alcohol alkoxylates of the formulae (I), (Ia), (II) and (IIb).

The alcohol alkoxylates of the formula (X) according to the invention also have a hydrophobic moiety (—(C₁₀H₂₀O)ₓ—Z) and a hydrophilic moiety (—(CₘH₂ₘO)_{q}—(C₂H₄O)_{z}—).

In accordance with a particular embodiment, the hydrophobic moiety of the alcohol alkoxylates according to the invention comprises at least 15 carbon atoms in the radical Z and in the group —(C₁₀H₂₀O)ₓ— (in other words, the total of (10 ·x) and the number of the carbon atoms in Z is at least 15). Preferably, the hydrophobic moiety of the alcohol alkoxylates according to the invention has at least 17 and in particular at least 19 carbon atoms. On the other hand, the hydrophobic moiety of the alcohol alkoxylates has, in a further aspect of the invention, not more than 60, preferably not more than 55 or 50 and in particular not more than 46 carbon atoms.

In accordance with a further particular embodiment, the hydrophobic moiety of the alcohol alkoxylates of the formula (X) according to the invention comprises at least 2 branches. Here, at least 1 branch is located in the alkoxylate moiety. On the other hand, the hydrophobic moiety of the alcohol alkoxylates comprises, in a further aspect of the invention, not more than 12, preferably not more than 11 or 10, and in particular not more than 9 branches. In fact, each decylene oxide contributes one branch, which is why the number of branches in the hydrophobic alkoxylate moiety corresponds to the total of the carbon atoms contributed by the decylene oxide units, i.e. (10 ·x).

In accordance with a further particular embodiment, the hydrophobic moiety of the alcohol alkoxylates of the formula (X) according to the invention comprises at least approximately 0.1, preferably at least approximately 0.13 and in particular at least approximately 0.16 branches per C atom. On the other hand, the hydrophobic moiety of the alcohol alkoxylates comprises, in a further aspect of the invention, not more than 0.3, preferably not more than 0.27 and in particular not more than 0.25 branches.

In a further particular aspect of the invention, the alcohol alkoxylates of the formula (X) according to the invention have at least approximately 1 or more, preferably at least approximately 1.5 or more, in particular at least approximately 2 or more decylene oxide units (value of x). On the other hand, the alcohol alkoxylates of the formula (X) according to the invention have, in a further particular aspect of the invention, not more than approximately 5 or less, preferably not more than approximately 4 or less and in particular not more than approximately 3 or less decylene oxide units (value of x). Accordingly, preferred alcohol alkoxylates of the formula (X) are those which comprise approximately 1 to 5, preferably approximately 1.5 to 4 and in particular approximately 2 to 3 decylene oxide units (value of x).

In a further particular aspect of the invention, the alcohol alkoxylates of the formula (X) according to the invention have at least approximately 2 or more, preferably at least approximately 3 or more, in particular at least approximately 5 or more ethylene oxide units (value of z). On the other hand, the alcohol alkoxylates of the formula (X) according to the invention have, in a further particular aspect of the invention, not more than approximately 30 or less, preferably not more than approximately 20 or less and in particular not more than approximately 12 or less ethylene oxide units (value of z). Accordingly, preferred alcohol alkoxylates of the formula (X) are those which comprise approximately 2 to 30, preferably approximately 3 to 20 and in particular approximately 5 to 12 ethylene oxide units (value of z).

In a further particular aspect of the invention, the alcohol alkoxylates of the formula (X) according to the invention have an alkoxylate moiety in which the ratio of ethylene oxide to decylene oxide (z to x) is at least 1:1 or more, preferably at least 3:1 or more, and in particular at least 5:1 or more. On the other hand, the alcohol alkoxylates of the formula (X) according to the invention have, in a further particular aspect of the invention, an alkoxylate moiety in which the ratio of ethylene oxide to decylene oxide (z to x) is not more than 30:1 or less, preferably not more than 15:1 or less, and in particular not more than 10:1 or less. Accordingly, preferred alkoxylates of the formula (X) are those in which the ratio of ethylene oxide to decylene oxide (z to x) is 1:1 to 30:1, preferably 3:1 to 15:1 and in particular 5:1 to 10:1.

One particular embodiment is alcohol alkoxylates according to the invention which are not end-group capped (Z═H).

In accordance with a further preferred embodiment, the alcohol alkoxylates according to the invention are end-group capped. In this case, Z preferably represents C₁-C₄-alkyl, more preferably C₁-C₃-alkyl and in particular methyl. Other radicals which are furthermore suitable for Z are C₂-C₄-alkenyl (for example allyl), C₆-C₁₀-aryl (for example phenyl) or C₆-C₁₀-aryl-C₁-C₂-alkyl (for example benzyl), C₁-C₄-alkylcarbonyl (for example acetyl, propionyl, butyryl), C₆-C₁₀-arylcarbonyl (for example benzoyl). Tertiary alcohol residues such as 2-hydroxyisobutyl or inorganic acid groups, in particular phosphate, diphosphate or sulfate, are also suitable.

End-group capped alcohol alkoxylates can be prepared in a manner known per se by reacting the non-end-group capped alcohol alkoxylate with suitable reagents, for example dialkyl sulfates. Such reactions are described for example in EP-A 0 302 487 and EP-A 0 161 537, whose disclosure is herewith incorporated in its entirety by reference.

The theoretical molecular weight of alcohol alkoxylates which are suitable in accordance with the invention is, as a rule, less than 2000 g/mol. Preferred are alcohol alkoxylates with a molecular weight of less than 1800 g/mol, less than 1700 g/mol, or less than 1500 g/mol. In accordance with a particular embodiment, the molecular weight is less than 1400 g/mol.

The weight-average molecular weight of alcohol alkoxylates which are suitable in accordance with the invention is, as a rule, less than 2000 g/mol. Preferred are alcohol alkoxylates with a molecular weight of less than 1800 g/mol, less than 1700 g/mol, or less than 1500 g/mol. In accordance with a particular embodiment, the molecular weight is less than 1400 g/mol. The weight-average molecular weight data relate to the determination by means of gel permeation chromatography according to DIN 55672.

The term “degree of branching” of R is here defined in a manner which is known in principle as the number of methyl groups in R minus 1. The same applies analogously to Z. The degree of branching of the alkoxylate moiety is calculated from the degree of alkoxylation and the alkylene oxides involved in the alkoxylation. The mean degree of branching is the statistic mean of the degrees of branching of all molecules of a sample.

The mean degree of branching can be determined by ¹H NMR spectroscopy as shown hereinbelow for primary and/or secondary alcohols: To this end, a sample of the alcohol is first derivatized with trichloroacetyl isocyanate (TAI). In this process, the alcohols are converted into the carbamic esters. The signals of the esterified primary alcohols are at δ=4.7 to 4.0 ppm, those of the esterified secondary alcohols at approximately 5 ppm, and water present in the sample reacts with TAI to give carbamic acid. All methyl, methylene and methyne protons are in the range of from 2.4 to 0.4 ppm. The signals<1 ppm are assigned to the methyl groups. The mean degree of branching (iso-index) can be calculated from the spectrum thus obtained as follows:

iso-index=((F(CH₃)/3)/(F(CH₂—OH)/2+F(CHR—OH)))−1

where F(CH₃) is the signal area which corresponds to the methyl protons, F(CH₂—OH) the signal area of the methylene protons in the CH₂—OH group and F(CHR—OH) the signal area of the methyne protons in the CHR—OH group.

Amounts of component (b), i.e. of alcohol alkoxylate based on the total weight of the composition according to the invention of more than 1% by weight, preferably of more than 5% by weight and in particular of more than 10% by weight, are advantageous. On the other hand, amounts of component (b) based on the total weight of the composition of less than 50% by weight, preferably less than 45% by weight and in particular less than 40% by weight, are, as a rule, expedient.

The plant treatment agent of component (a) can mean any substance whose purpose or effect it is to prevent the attack of any pest on a plant, or to safeguard against, repel or destroy the pest, or to reduce the damage caused by it in any other manner (pesticide). As has been stated at the outset, plant pests may belong to different groups of organisms; among the higher animals, a large number of important pests can be found in particular among the insects and mites, furthermore among nematodes and slugs and snails; vertebrates such as mammals and birds are of lesser importance in today's industrialized countries. A large number of groups of microbes, among which fungi, bacteria including the mycoplasmata, viruses and viroids comprise pests, and also weeds, which compete with useful plants for scarce living space and other resources, can be included among the pests in the wider sense. Pesticides comprise in particular avicides, acaricides, desiccants, bactericides, chemosterilants, defoliants, antifeedants, fungicides, herbicides, herbicide safeners, insect attractants, insecticides, insect repellants, molluscides, nematicides, mating disruptors, plant activators, plant growth regulators, rodenticides, mammalian repellents, synergists, bird repellents and virucides.

Divided by chemical classes, pesticides comprise, in particular acylalanine fungicides, acylamino acid fungicides, aliphatic amide-organothiophosphate insecticides, aliphatic organothiophosphate insecticides, aliphatic nitrogen fungicides, amide fungicides, amide herbicides, anilide fungicides, anilide herbicides, inorganic fungicides, inorganic herbicides, inorganic rodenticides, antiauxins, antibiotic acaricides, antibiotic fungicides, antibiotic herbicides, antibiotic insecticides, antibiotic nematicides, aromatic acid fungicides, aromatic acid herbicides, arsenic herbicides, arsenic insecticides, arylalanine herbicides, aryloxyphenoxypropionic acid herbicides, auxins, avermectin acaricides, avermectin insecticides, benzamide fungicides, benzanilide fungicides, benzimidazole fungicides, benzimidazole precursor fungicides, benzimidazolylcarbamate fungicides, benzoic acid herbicides, benzofuranyl alkylsulfonate herbicides, benzofuranyl methylcarbamate insecticides, benzothiazole fungicides, benzothiopyran-organothiophosphate insecticides, benzotriazine-organothiophosphate insecticides, benzoylcyclohexanedione herbicides, bipyridylium herbicides, bridge diphenyl acaricides, bridge diphenyl fungicides, carbamate acaricides, carbamate fungicides, carbamate herbicides, carbamate insecticides, carbamate nematicides, carbanilate fungicides, carbanilate herbicides, quinolinecarboxylate herbicides, quinoline fungicides, quinone fungicides, quinoxaline acaricides, quinoxaline-organothio-phosphate insecticides, quinoxaline fungicides, chitin synthesis inhibitors, chloroacetanilide herbicides, chloronicotinyl insecticides, chloropyridine herbicides, chlorotriazine herbicides, conazole fungicides, coumarin rodenticides, cyclodithiocarbamate fungicides, cyclohexene oxime herbicides, cyclopropylisoxazole herbicides, cytokinins, diacylhydrazine insecticides, dicarboximide fungicides, dicarboximide herbicides, dichlorophenyldicarboximide fungicides, dimethylcarbamate insecticides, dinitroaniline herbicides, dinitrophenol acaricides, dinitrophenol fungicides, dinitrophenol herbicides, dinitrophenol insecticides, diphenyl ether herbicides, dithiocarbamate fungicides, dithiocarbamate herbicides, defoliants, ethylene-releasing agents, fluorine insecticides, furamide fungicides, furanilide fungicides, gibberellins, halogenated aliphatic herbicides, urea fungicides, urea herbicides, urea insecticides, urea rodenticides, molting hormones, molting hormone mimetics, molting inhibitors, heterocyclic organothiophosphate insecticides, imidazole fungicides, imidazolinone herbicides, indandione rodenticides, insect growth regulators, isoindole-organothio-phosphate insecticides, isoxazole-organothiophosphate insecticides, juvenile hormones, juvenile hormone mimetics, copper fungicides, macrocyclic lactone acaricides, macrocyclic lactone insecticides, methoxytriazine herbicides, methylthiotriazine herbicides, milbemycin acaricides, milbemycin insecticides, mite growth regulators, morphactins, morpholine fungicides, nereistoxin analogs, nicotinoid insecticides, nitrile herbicides, nitroguanidine insecticides, nitromethylene insecticides, nitrophenyl ether herbicides, organochlorine acaricides, organochlorine insecticides, organochlorine rodenticides, organophosphate acaricides, organophosphate insecticides, organophosphate nematicides, organophosphorus acaricides, organophosphorus fungicides, organophosphorus herbicides, organophosphorus insecticides, organophosphorus nematicides, organophosphorus rodenticides, organothiophosphate acaricides, organothiophosphate insecticides, organothiophosphate nematicides, organotin acaricides, organotin fungicides, oxadiazine insecticides, oxathine fungicides, oxazole fungicides, oxime carbamate acaricides, oxime carbamate nematicides, oxime carbamate insecticides, oxime-organothiophosphate insecticides, plant-based insecticides, plant-based rodenticides, phenoxybutyric acid herbicides, phenoxyacetic acid herbicides, phenoxy herbicides, phenoxypropionic acid herbicides, phenylenediamine herbicides, phenylethyl phosphonothioate insecticides, phenylurea herbicides, phenylmethylcarbamate insecticides, phenylorganothiophosphate insecticides, phenylphenylphosphonothioate insecticides, phenyl pyrazolyl ketone herbicides, phenylsulfamide acaricides, phenylsulfamide fungicides, phosphonate acaricides, phosphonate insecticides, phosphonothioate insecticides, phosphoramidate insecticides, phosphoramidothioate acaricides, phosphoramidothioate insecticides, phosphorus diamide acaricides, phosphorus diamide insecticides, phthalate herbicides, phthalimide acaricides, phthalimide fungicides, phthalimide insecticides, picolate herbicides, polymeric dithiocarbamate fungicides, polysulfide fungicides, precocenes, pyrazole acaricides, pyrazole fungicides, pyrazole insecticides, pyrazolopyrimidine-organothiophosphate insecticides, pyrazolyloxyacetophenone herbicides, pyrazolylphenyl herbicides, pyrethroid acaricides, pyrethroid ester acaricides, pyrethroid ester insecticides, pyrethroid ether acaricides, pyrethroid ether insecticides, pyrethroid insecticides, pyridazine herbicides, pyridazinone herbicides, pyridine fungicides, pyridine herbicides, pyridine-organothiophosphate insecticides, pyridylmethylamine insecticides, pyrimidinamine acaricides, pyrimidinamine insecticides, pyrimidinamine rodenticides, pyrimidinediamine herbicides, pyrimidine-organothiophosphate insecticides, pyrimidine fungicides, pyrimidinyloxy benzoic acid herbicides, pyrimidinylsulfonylurea herbicides, pyrimidinylthiobenzoic acid herbicides, pyrrole acaricides, pyrrole fungicides, pyrrole insecticides, quaternary ammonium herbicides, strobilurin fungicides, sulfite ester acaricides, sulfonamide fungicides, sulfonamide herbicides, sulfonanilide fungicides, sulfonanilide herbicides, sulfonylurea herbicides, tetrazine acaricides, tetronate acaricides, tetronate insecticides, thiadiazole-organothiophosphate insecticides, thiadiazolylurea herbicides, thiazole fungicides, thiocarbamate acaricides, thiocarbamate fungicides, thiocarbamate herbicides, thiocarbonate herbicides, thiourea acaricides, thiourea herbicides, thiourea rodenticides, thiophene fungicides, triazine fungicides, triazine herbicides, triazinone herbicides, triazinylsulfonylurea herbicides, triazole fungicides, triazole herbicides, triazolone herbicides, triazolopyrimidine fungicides, triazolopyrimidine herbicides, triazole-organothiophosphate insecticides, uracil herbicides, valinamide fungicides, growth inhibitors, growth stimulators, growth retardants, xylylalanine fungicides.

The pesticide for use according to the invention is selected in particular among fungicides (a1), herbicides (a2) and insecticides (a3).

Fungicides comprise, for example, aliphatic nitrogen fungicides, such as butylamine, cymoxanil, dodicin, dodine, guazatine, iminoctadine; amide fungicides, such as carpropamid, chloraniformethan, cyflufenamid, diclocymet, ethaboxam, fenoxanil, flumetover, furametpyr, mandipropamid, penthiopyrad, prochloraz, chinazamid, silthiofam, triforine; in particular acylamino acid fungicides, such as benalaxyl, benalaxyl-M, furalaxyl, metalaxyl, metalaxyl-M, pefurazoate; anilide fungicides, such as benalaxyl, benalaxyl-M, boscalid, carboxin, fenhexamid, metalaxyl, metalaxyl-M, metsulfovax, ofurace, oxadixyl, oxycarboxin, pyracarbolid, thifluzamide, tiadinil; in particular benzanilide fungicides, such as benodanil, flutolanil, mebenil, mepronil, salicylanilides, tecloftalam; furanilide fungicides, such as fenfuram, furalaxyl, furcarbanil, methfuroxam; and sulfonanilide fungicides, such as flusulfamide; benzamide fungicides, such as benzohydroxamic acid, fluopicolide, tioxymid, trichlamide, zarilamid, zoxamide; furamide fungicides, such as cyclafuramid, furmecyclox; phenylsulfamide fungicides, such as dichlofluanid, tolylfluanid; sulfonamide fungicides, such as cyazofamid; and valinamide fungicides, such as benthiavalicarb, iprovalicarb; antibiotic fungicides, such as aureofungin, blasticidin-S, cycloheximide, griseofulvin, kasugamycin, natamycin, polyoxins, polyoxorim, streptomycin, validamycin; in particular strobilurin fungicides, such as azoxystrobin, dimoxystrobin, fluoxastrobin, kresoxim-methyl, metominostrobin, orysastrobin, picoxystrobin, pyraclostrobin, trifloxystrobin; aromatic fungicides, such as biphenyl, chlorodinitronaphthalene, chloroneb, chlorothalonil, cresol, dicloran, quintozene, tecnazene; benzimidazole fungicides, such as benomyl, carbendazim, chlorfenazole, cypendazole, debacarb, fuberidazole, mecarbinzid, rabenzazole, thiabendazole; benzimidazole precursor fungicides, such as furophanate, thiophanate, thiophanate methyl; benzothiazole fungicides, such as bentaluron, chlobenthiazon, TCMTB; bridge diphenyl fungicides, such as bithionol, dichlorophen, diphenylamine; carbamate fungicides, such as benthiavalicarb, furophanate, iprovalicarb, propamocarb, thiophanate, thiophanate-methyl; in particular benzimidazolylcarbamate fungicides, such as benomyl, carbendazim, cypendazole, debacarb, mecarbinzid; and carbanilate fungicides, such as diethofencarb; conazole fungicides; in particular imidazoles, such as climbazole, clotrimazole, imazalil, oxpoconazole, prochloraz, triflumizole; and triazoles, such as azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-p; copper fungicides, such as Bordeaux mixture, Burgundy mixture, Cheshunt mixture, copper acetate, copper carbonate, copper hydroxide, copper naphthenate, copper oleate, copper oxychloride, copper sulfate, copper zinc chromate, copper oxide, mancopper, cufraneb, cuprobam, oxine-copper; dicarboximide fungicides, such as famoxadon, fluoroimide; in particular dichlorophenyldicarboximide fungicides, such as chlozolinate, dichlozoline, iprodion, isovaledion, myclozolin, procymidon, vinclozolin; and phthalimide fungicides, such as captafol, captan, ditalimfos, folpet, thiochlorfenphim; dinitrophenol fungicides, such as binapacryl, dinobuton, dinocap, dinocap-4, dinocap-6, dinocton, dinopenton, dinosulfon, dinoterbon, DNOC; dithiocarbamate fungicides, such as azithiram, carbamorph, cufraneb, cuprobam, disulfuram, ferbam, metam, nabam, tecoram, thiram, ziram; in particular cyclodithiocarbamate fungicides, such as dazomet, etem, milneb; and polymeric dithiocarbamate fungicides, such as mancopper, mancozeb, maneb, metiram, polycarbamate, propineb, zineb; imidazole fungicides, such as cyazofamid, fenamidon, fenapanil, glyodin, iprodione, isovaledion, pefurazoate, triazoxide; inorganic fungicides, such as potassium azide, sodium azide, sulfur; morpholine fungicides, such as, for example, aldimorph, benzamorph, carbamorph, dimethomorph, dodemorph, fenpropimorph, flumorph, tridemorph; organophosphorus fungicides, such as ampropylfos, ditalimfos, edifenphos, fosetyl, hexylthiofos, iprobenfos, phosdiphen, pyrazophos, tolclofos-methyl, triamiphos; organotin fungicides, such as decafentin, fentin, tributyltin oxide; oxathine fungicides, such as carboxin, oxycarboxin; oxazole fungicides, such as chlozolinate, dichlozoline, drazoxolon, famoxadon, hymexazol, metazoxolon, myclozolin, oxadixyl, vinclozolin; polysulfide fungicides, such as barium polysulfide, potassium polysulfide, sodium polysulfide; pyrazole fungicides, such as furametpyr, penthiopyrad; pyridine fungicides, such as boscalid, buthiobate, dipyrithion, fluazinam, fluopicolide, pyridinitril, pyrifenox, pyroxychlor, pyroxyfur; pyrimidine fungicides, such as bupirimate, cyprodinil, diflumetorim, dimethirimol, ethirimol, fenarimol, ferimzon, mepanipyrim, nuarimol, pyrimethanil, triarimol; pyrrole fungicides, such as fenpiclonil, fludioxonil, fluoroimide; quinoline fungicides, such as ethoxyquin, halacrinate, 8-hydroxyquinoline sulfate, quinacetol, quinoxyfen; quinone fungicides, such as benquinox, chloranil, dichlon, dithianon; quinoxaline fungicides, such as quinomethionate, chlorquinox, thioquinox; thiazole fungicides, such as ethaboxam, etridiazole, metsulfovax, octhilinone, thiabendazole, thiadifluor, thifluzamide; thiocarbamate fungicides, such as methasulfocarb, prothiocarb; thiophene fungicides, such as ethaboxam, silthiofam; triazine fungicides, such as anilazine; triazole fungicides, such as bitertanol, fluotrimazole, triazbutil; urea fungicides, such as bentaluron, pencycuron, quinazamid; unclassified fungicides, such as acibenzolar, acypetacs, allyl alcohol, benzalkonium chloride, benzamacril, bethoxazin, carvone, DBCP, dehydroacetic acid, diclomezine, diethyl pyrocarbonate, fenaminosulf, fenitropan, fenpropidin, formaldehyde, furfural, hexachlorobutadiene, isoprothiolane, methyl isothiocyanate, metrafenon, nitrostyrene, nitrothal-isopropyl, OCH, phthalide, piperalin, probenazole, proquinazid, pyroquilon, sodium orthophenyl phenoxide, spiroxamine, sultropen, thicyofen, tricyclazole, zinc naphthenate.

In accordance with a particular embodiment of the invention, fungicides (a1) comprise:

acylalanines, such as benalaxyl, metalaxyl, ofurace, oxadixyl;
amine derivatives, such as aldimorph, dodine, dodemorph, fenpropimorph, fenpropidin, guazatine, iminoctadine, spiroxamine, tridemorph;
anilinopyrimidines, such as pyrimethanil, mepanipyrimorcyprodinil;
antibiotics, such as cycloheximide, griseofulvin, kasugamycin, natamycin, polyoxin and streptomycin;
azoles: azaconazole, bitertanol, bromoconazole, cyproconazole, dichlobutrazol, difenoconazole, dinitroconazole, epoxiconazole, fenbuconazole, fluquinconazole, flusilazole, flutriafol, ketoconazole, hexaconazole, metconazole, myclobutanile, penconazole, propiconazole, prothioconazole, tebuconazole, tetraconazole, triadimefone, triadimenol, triflumizole, triticonazole;
dicarboximides, such as iprodione, myclozolin, procymidon, vinclozolin; dithiocarbamates: ferbam, nabam, maneb, mancozeb, metam, metiram, propineb, polycarbamate, thiram, ziram, zineb;
heterocylic compounds, such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophenate-methyl, tiadinil, tricyclazole, triforine;
nitrophenyl derivatives, such as binapacryl, dinocap, dinobuton, nitrophthal-isopropyl; phenylpyrroles, such as fenpiclonil and fludioxonil;
2-methoxybenzophenones as described in EP-A897904, for example metrafenone; fungicides which do not belong to any other class, such as acibenzolar-5-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, metrafenon, pencycuron, propamocarb, phthalide, toloclofos-methyl, quintozene, zoxamide; strobilurins as described in WO03/075663, for example azoxystrobin, dimoxystrobin, fluoxastrobin, cresoxim-methyl, metominostrobin, orysastrobin, picoxystrobin, pyraclostrobin and trifloxystrobin;
sulfonates, such as captafol, captan, dichlofluanid, folpet, tolylfluanid;
cinnamides and their analogs, such as dimethomorph, flumetover, flumorph;
6-aryl[1,2,4]triazole[1,5-a]-pyrimidines as described for example in WO98/46608, WO99/41255 or WO03/004465, for example 5-chloro-7-(4-methylpiperidin-1-yl)-6-(2,4,6-trifluorophenyl)-[1,2,4]triazole[1,5-a]-pyrimidine, 5-chloro-7-(4-methylpiperazin-1-yl)-6-(2,4,6-trifluorophenyl)-[1,2,4]triazole[1,5-a]-pyrimidine, 5-chloro-7-(morpholin-1-yl)-6-(2,4,6-trifluorophenyl)-[1,2,4]-triazole[1,5-a]-pyrimidine, 5-chloro-7-(piperidin-1-yl)-6-(2,4,6-trifluorophenyl)-[1,2,4]triazole[1,5-a]-pyrimidine, 5-chloro-7-(morpholin-1-yl)-6-(2,4,6-trifluorophenyl)-[1,2,4]triazole[1,5-a]-pyrimidine, 5-chloro-7-(isopropylamino)-6-(2,4,6-trifluorophenyl)-[1,2,4]triazole[1,5-a]-pyrimidine, 5-chloro-7-(cyclopentylamino)-6-(2,4,6-trifluorophenyl)-[1,2,4]triazole[1,5-a]-pyrimidine, 5-chloro-7-(2,2,2-trifluoroethyl-amino)-6-(2,4,6-trifluorophenyl)-[1,2,4]triazole[1,5-a]-pyrimidine, 5-chloro-7-(1,1,1-trifluoropropan-2-ylamino)-6-(2,4,6-trifluorophenyl)-[1,2,4]triazole[1,5-a]-pyrimidine, 5-chloro-7-(3,3-dimethylbutan-2-ylamino)-6-(2,4,6-trifluorophenyl)-[1,2,4]triazole[1,5-a]-pyrimidine, 5-chloro-7-(cyclohexylmethyl)-6-(2,4,6-trifluorophenyl)-[1,2,4]triazole[1,5-a]-pyrimidine, 5-chloro-7-(cyclohexyl)-6-(2,4,6-trifluorophenyl)-[1,2,4]triazole[1,5-a]-pyrimidine, 5-chloro-7-(2-methylbutan-3-yl)-6-(2,4,6-trifluorophenyl)-[1,2,4]triazole[1,5-a]-pyrimidine, 5-chloro-7-(3-methylpropan-1-yl)-6-(2,4,6-trifluorophenyl)-[1,2,4]triazole[1,5-a]-pyrimidine, 5-chloro-7-(4-methylcyclohexan-1-yl)-6-(2,4,6-trifluorophenyl)-[1,2,4]triazole[1,5-a]-pyrimidine, 5-chloro-7-(hexan-3-yl)-6-(2,4,6-trifluorophenyl)-[1,2,4]triazole[1,5-a]-pyrimidine, 5-chloro-7-(2-methylbutan-1-yl)-6-(2,4,6-trifluorophenyl)-[1,2,4]triazole[1,5-a]-pyrimidine, 5-chloro-7-(3-methylbutan-1-yl)-6-(2,4,6-trifluorophenyl)-[1,2,4]triazole[1,5-a]-pyrimidine, 5-chloro-7-(1-methylpropan-1-yl)-6-(2,4,6-trifluorophenyl)-[1,2,4]triazole[1,5-a]-pyrimidine, 5-methyl-7-(4-methyl-piperidin-1-yl)-6-(2,4,6-trifluorophenyl)-[1,2,4]triazole[1,5-a]-pyrimidine, 5-methyl-7-(4-methylpiperazin-1-yl)-6-(2,4,6-trifluorophenyl)-[1,2,4]triazole[1,5-a]-pyrimidine, 5-methyl-7-(morpholin-1-yl)-6-(2,4,6-trifluorophenyl)-[1,2,4]-triazole[1,5-a]-pyrimidine, 5-methyl-7-(piperidin-1-yl)-6-(2,4,6-trifluorophenyl)-[1,2,4]triazole[1,5-a]-pyrimidine, 5-methyl-7-(morpholin-1-yl)-6-(2,4,6-trifluorophenyl)-[1,2,4]triazole[1,5-a]-pyrimidine, 5-methyl-7-(isopropylamino)-6-(2,4,6-trifluorophenyl)-[1,2,4]triazole[1,5-a]-pyrimidine, 5-methyl-7-(cyclopentylamino)-6-(2,4,6-trifluorophenyl)-[1,2,4]triazole[1,5-a]-pyrimidine, 5-methyl-7-(2,2,2-trifluoroethylamino)-6-(2,4,6-trifluorophenyl)-[1,2,4]triazole[1,5-a]-pyrimidine, 5-methyl-7-(1,1,1-trifluoropropan-2-ylamino)-6-(2,4,6-trifluorophenyl)-[1,2,4]triazole[1,5-a]-pyrimidine, 5-methyl-7-(3,3-dimethylbutan-2-yl-amino)-6-(2,4,6-trifluorophenyl)-[1,2,4]triazole[1,5-a]-pyrimidine, 5-methyl-7-(cyclohexylmethyl)-6-(2,4,6-trifluorophenyl)-[1,2,4]-triazole[1,5-a]-pyrimidine, 5-methyl-7-(cyclohexyl)-6-(2,4,6-trifluorophenyl)-[1,2,4]triazole[1,5-a]-pyrimidine, 5-methyl-7-(2-methylbutan-3-yl)-6-(2,4,6-trifluorophenyl)-[1,2,4]triazole[1,5-a]-pyrimidine, 5-methyl-7-(3-methylpropan-1-yl)-6-(2,4,6-trifluorophenyl)-[1,2,4]triazole[1,5-a]-pyrimidine, 5-methyl-7-(4-methylcyclohexan-1-yl)-6-(2,4,6-trifluorophenyl)-[1,2,4]triazole[1,5-a]-pyrimidine, 5-methyl-7-(hexan-3-yl)-6-(2,4,6-trifluorophenyl)-[1,2,4]triazole[1,5-a]-pyrimidine, 5-methyl-7-(2-methylbutan-1-yl)-6-(2,4,6-trifluorophenyl)-[1,2,4]triazole[1,5-a]-pyrimidine, 5-methyl-7-(3-methylbutan-1-yl)-6-(2,4,6-trifluorophenyl)-[1,2,4]triazole[1,5-a]-pyrimidine and 5-methyl-7-(1-methylpropan-1-yl)-6-(2,4,6-trifluorophenyl)-[1,2,4]triazole[1,5-a]-pyrimidine;
amide fungicides, such as cyclofenamid, and (Z)—N—[a-(cyclopropylmethoxyimino)-2,3-difluoro-6-(difluoromethoxy)benzyl]-2-phenylacetamide.

Herbicides (a2) comprise, for example, amide herbicides, such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid, tebutam; in particular anilide herbicides, such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen, propanil; in particular arylalanine herbicides, such as benzoylprop, flamprop, flamprop-M; chloroacetanilide herbicides, such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor, xylachlor; and sulfonanilide herbicides, such as benzofluor, cloransulam, diclosulam, florasulam, flumetsulam, metosulam, perfluidon, pyrimisulfan, profluazole; and sulfonamide herbicides, such as asulam, carbasulam, fenasulam, oryzalin, penoxsulam; antibiotic herbicides, such as bilanafos; aromatic acid herbicides; in particular benzoate herbicides, such as chloramben, dicamba, 2,3,6-TBA, tricamba; in particular pyrimidinyloxybenzoate herbicides, such as bispyribac, pyriminobac; and pyrimidinylthiobenzoate herbicides, such pyrithiobac; phthalate herbicides, such as chlorthal; picolinate herbicides, such as aminopyralid, clopyralid, picloram; and quinolinecarboxylate herbicides, such as quinclorac, quinmerac; arsenic herbicides, such as cacodylate, CMA, DSMA, hexaflurate, MAA, MAMA, MSMA, potassium arsenite, sodium arsenite; benzoylcyclohexanedione herbicides, such as mesotrione, sulcotrione; benzofuranylalkylsulfonate herbicides, such as benfuresate, ethofumesate; carbamate herbicides, such as asulam, carboxazole, chlorprocarb, dichlormat, fenasulam, karbutilate, terbucarb; carbanilate herbicides, such as barbane, BCPC, carbasulam, carbetamid, CEPC, chlorbufam, chiorpropham, CPPC, desmedipham, phenisopham, phenmedipham, phenmedipham-ethyl, propham, swep; cyclohexene oxime herbicides, such as alloxydim, butroxydim, clethodim, cloproxydim, cycloxydim, profoxydim, sethoxydim, tepraloxydim, tralkoxydim; cyclopropylisoxazole herbicides, such as isoxachlortol, isoxaflutol; dicarboximide herbicides, such as benzfendizon, cinidon-ethyl, flumezin, flumiclorac, flumioxazin, flumipropyne; dinitroaniline herbicides, such as benfluralin, butralin, dinitramine, ethalfluralin, fluchloralin, isopropalin, methalpropalin, nitralin, oryzalin, pendimethalin, prodiamine, profluralin, trifluralin; dinitrophenol herbicides, such as dinofenat, dinoprop, dinosam, dinoseb, dinoterb, DNOC, etinofen, medinoterb; diphenyl ether herbicides, such as ethoxyfen; in particular nitrophenyl ether herbicides, such as acifluorfen, aclonifen, bifenox, chlomethoxyfen, chiornitrofen, etnipromid, fluorodifen, fluoroglycofen, fluoronitrofen, fomesafen, furyloxyfen, halosafen, lactofen, nitrofen, nitrofluorfen, oxyfluorfen; dithiocarbamate herbicides, such as dazomet, metam; haloaliphatic herbicides, such as alorac, chloropon, dalapon, flupropanate, hexachloroacetone, chloroacetic acid, SMA, TCA; imidazolinone herbicides, such as imazamethabenz, imazamox, imazapic, imazapyr, imazaquin, imazethapyr; inorganic herbicides, such as ammonium sulfamate, calcium chlorate, copper sulfate, iron sulfate, potassium azide, pottassium cyanide, sodium azide, sodium chlorate, sulfuric acid; nitrile herbicides, such as bromobonil, bromoxynil, chloroxynil, dichlobenil, iodobonil, ioxynil, pyraclonil; organophosphorus herbicides, such as amiprofos-methyl, anilofos, bensulide, bilanafos, butamifos, 2,4-DEP, DMPA, EBEP, fosamine, glufosinate, glyphosate, piperophos; phenoxy herbicides, such as bromofenoxim, clomeprop, 2,4-DEB, 2,4-DEP, difenopentene, disul, erbon, etnipromid, fenteracol, trifopsime; in particular phenoxyacetic acid herbicides, such as 4-CPA, 2,4-D, 3,4-DA, MCPA, MCPA-thioethyl; phenoxybutyric acid herbicides, such as 4-CPB, 2,4-DB, 3,4-DB, MCPB, 2,4,5-TB; and phenoxypropionic acid herbicides, such cloprop, 4-CPP, dichlorprop, dichlorprop-P, 3,4-DP, fenoprop, mecoprop, mecoprop-P; in particular aryloxyphenoxypropionic acid herbicides, such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P, trifop; phenylenediamine herbicides, such as dinitramine, prodiamine; phenyl pyrazolyl ketone herbicides, such as benzofenap, pyrazolynate, pyrazoxyfen, topramezone; pyrazolylphenyl herbicides, such as fluazolate, pyraflufen; pyridazine herbicides, such as credazin, pyridafol, pyridate; pyridazinone herbicides, such as brompyrazon, chloridazon, dimidazon, flufenpyr, metfiurazon, norflurazon, oxapyrazon, pydanon; pyridine herbicides, such as aminopyralid, cliodinate, clopyralid, dithiopyr, fluoroxypyr, haloxydine, picloram, picolinafen, pyriclor, thiazopyr, triclopyr; pyrimidinediamine herbicides, such as iprymidam, tioclorim; quaternary ammonium herbicides, such as cyperquat, diethamquat, difenzoquat, diquat, morfamquat, paraquat; thiocarbamate herbicides, such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate, vernolate; thiocarbonate herbicides, such as dimexano, EXD, proxan; thiourea herbicides, such as methiuron; triazine herbicides, such as dipropetryne, triaziflam, trihydroxytriazine; in particular chlorotriazine herbicides, such as atrazine, chlorazine, cyanazine, cyprazine, eglinazine, ipazine, mesoprazine, procyazine, proglinazine, propazine, sebuthylazine, simazine, terbuthylazine, trietazine; methoxytriazine herbicides, such as atraton, methometon, prometon, secbumeton, simeton, terbumeton; and methylthiotriazine herbicides, such as ametryn, aziprotryne, cyanatryn, desmetryn, dimethametryn, methoprotryne, prometryn, simetryn, terbutryne; triazinone herbicides, such as ametridione, amibuzin, hexazinone, isomethiozin, metamitron, metribuzin; triazole herbicides, such as amitrole, cafenstrol, epronaz, flupoxam; triazolone herbicides, such as amicarbazone, carfentrazone, flucarbazone, propoxycarbazone, sulfentrazone; triazolopyrimidine herbicides, such as cloransulam, diclosulam, florasulam, flumetsulam, metosulam, penoxsulam; uracil herbicides, such as butafenacil, bromacil, flupropacil, isocil, lenacil, terbacil; urea herbicides, such as benzthiazuron, cumyluron, cycluron, dichloral urea, diflufenzopyr, isonoruron, isouron, methabenzthiazuron, monisouron, noruron; in particular phenylurea herbicides, such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluoron, phenobenzuron, siduron, tetrafluoron, thidiazuron; sulfonylurea herbicides; in particular pyrimidinylsulfonylurea herbicides, such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron, trifloxysulfuron; and triazinylsulfonylurea herbicides, such as chlorsulfuron, cinosulfuron, ethametsulfuron, iodosulfuron, metsulfuron, prosulfuron, thifensulfuron, triasulfuron, tribenuron, triflusulfuron, tritosulfuron; and thiadiazolylurea herbicides, such as buthiuron, ethidimuron, tebuthiuron, thiazafluoron, thidiazuron; and other herbicides, such as acrolein, allyl alcohol, azafenidin, benazolin, bentazon, benzobicyclon, buthidazole, calcium cyanamide, cambendichlor, chlorfenac, chlorfenprop, chlorflurazole, chlorflurenol, cinmethylin, clomazone, CPMF, cresol, orthodichlorobenzene, dimepiperate, endothal, fluoromidine, fluridon, fluorochloridon, flurtamon, fluthiacet, indanofan, methazole, methyl isothiocyanate, nipyraclofen, OCH, oxadiargyl, oxadiazon, oxaziclomefon, pentoxazon, pinoxaden, prosulfalin, pyribenzoxim, pyriftalid, quinoclamine, rhodethanil, sulglycapin, thidiazimin, tridiphane, trimeturon, tripropindan, tritac.

In accordance with a particular embodiment of the invention, herbicides (a2) comprise:

Lipid biosynthesis inhibitors, such as, for example, chlorazifop, clodinafop, clofop, cyhalofop, diclofop, fenoxaprop, fenoxaprop-p, fenthiaprop, fluazifop, fluazifop-P, haloxyfop, haloxyfop-P, isoxapyrifop, metamifop, propaquizafop, quizalofop, quizalofop-P, trifop, alloxydim, butroxydim, clethodim, cloproxydim, cycloxydim, profoxydim, sethoxydim, tepraloxydim, tralkoxydim, butylate, cycloate, diallate, dimepiperate, EPTC, esprocarb, ethiolate, isopolinate, methiobencarb, molinate, orbencarb, pebulate, prosulfocarb, sulfallate, thiobencarb, tiocarbazil, triallate, vernolate, benfuresate, ethofumesate and bensulid;
ALS inhibitors, such as, for example, amidosulfuron, azimsulfuron, bensulfuron, chlorimuron, chlorsulfuron, cinosulfuron, cyclosulfamuron, ethametsulfuron, ethoxysulfuron, flazasulfuron, flupyrsulfuron, foramsulfuron, halosulfuron, imazosulfuron, iodosulfuron, mesosulfuron, metsulfuron, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, imazamethabenz, imazamox, imazapic, imazapyr, imazaquin, imazethapyr, cloransulam, diclosulam, florasulam, flumetsulam, metosulam, penoxsulam, bispyribac, pyriminobac, propoxycarbazone, flucarbazone, pyribenzoxim, pyriftalid and pyrithiobac;
Photosynthesis inhibitors, such as, for example, atraton, atrazine, ametryn, aziprotryn, cyanazine, cyanatryn, chlorazine, cyprazine, desmetryn, dimethametryn, dipropetryn, eglinazine, ipazine, mesoprazine, methometon, methoprotryn, procyazine, proglinazine, prometon, prometryn, propazine, sebuthylazine, secbumeton, simazine, simeton, simetryn, terbumeton, terbuthylazine, terbutryn, trietazine, ametridione, amibuzin, hexazinone, isomethiozin, metamitron, metribuzin, bromacil, isocil, lenacil, terbacil, brompyrazon, chloridazon, dimidazon, desmedipham, phenisopham, phenmedipham, phenmedipham-ethyl, benzthiazuron, buthiuron, ethidimuron, isouron, methabenzthiazuron, monoisouron, tebuthiuron, thiazafluoron, anisuron, buturon, chlorbromuron, chloreturon, chlorotoluron, chloroxuron, difenoxuron, dimefuron, diuron, fenuron, fluometuron, fluothiuron, isoproturon, linuron, methiuron, metobenzuron, metobromuron, metoxuron, monolinuron, monuron, neburon, parafluoron, phenobenzuron, siduron, tetrafluoron, thidiazuron, cyperquat, diethamquat, difenzoquat, diquat, morfamquat, paraquat, bromobonil, bromoxynil, chloroxynil, iodobonil, ioxynil, amicarbazone, bromofenoxim, flumezin, methazole, bentazone, propanil, pentanochlor, pyridate and pyridafol;
Protoporphyrinogen-IX oxidase inhibitors, such as, for example, acifluorfen, bifenox, chlomethoxyfen, chlornitrofen, ethoxyfen, fluorodifen, fluoroglycofen, fluoronitrofen, fomesafen, furyloxyfen, halosafen, lactofen, nitrofen, nitrofluorfen, oxyfluorfen, fluazolate, pyraflufen, cinidon-ethyl, flumiclorac, flumioxazin, flumipropyn, fluthiacet, thidiazimin, oxadiazon, oxadiargyl, azafenidin, carfentrazone, sulfentrazone, pentoxazone, benzfendizone, butafenacil, pyraclonil, profluazol, flufenpyr, flupropacil, nipyraclofen and etnipromid;
Bleacher herbicides, such as, for example, metflurazon, norflurazon, flufenican, diflufenican, picolinafen, beflubutamid, fluridone, fluorochloridone, flurtamone, mesotrione, sulcotrione, isoxachlortole, isoxaflutole, benzofenap, pyrazolynate, pyrazoxyfen, benzobicyclon, amitrole, clomazone, aclonifen, 4-(3-trifluoromethylphenoxy)-2-(4-trifluoromethylphenyl)pyrimidine and also 3-heterocyclyl-substituted benzoyl derivatives of the formula II (see WO 96/26202, WO 97/41116, WO 97/41117 and WO 97/41118)

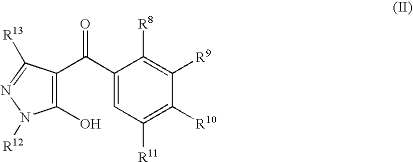

in which the variables R⁸ to R¹³ have the meanings below:
R⁸, R¹⁰ are hydrogen, halogen, C₁-C₆-alkyl, C₁-C₆-haloalkyl, C₁-C₆-alkoxy, C₁-C₆-haloalkoxy, C₁-C₆-alkylthio, C₁-C₆-alkylsulfinyl or C₁-C₆-alkyl-sulfonyl;
R⁹ is a heterocyclic radical selected from the group consisting of thiazol-2-yl, thiazol-4-yl, thiazol-5-yl, isoxazol-3-yl, isoxazol-4-yl, isoxazol-5-yl, 4,5-dihydroisoxazol-3-yl, 4,5-dihydroisoxazol-4-yl and 4,5-dihydroisoxazol-5-yl, where the nine radicals mentioned may be unsubstituted or mono- or polysubstituted, for example mono-, di-, tri- or tetrasubstituted, by halogen, C₁-C₄-alkyl, C₁-C₄-alkoxy, C₁-C₄-haloalkyl, C₁-C₄-haloalkoxy or C₁-C₄-alkylthio;
R¹¹ is hydrogen, halogen or C₁-C₆-alkyl;
R¹² is C₁-C₆-alkyl;
R¹³ is hydrogen or C₁-C₆-alkyl;
   EPSP synthase inhibitors, such as, for example, glyphosate;
   Glutamine synthase inhibitors, such as, for example glufosinate and bilanaphos;
   DHP synthase inhibitors, such as, for example asulam;
   Mitose inhibitors, such as, for example, benfluralin, butralin, dinitramine, ethalfluralin, fluchloralin, isopropalin, methalpropalin, nitralin, oryzalin, pendimethalin, prodiamin, profluralin, trifluralin, amiprofosmethyl, butamifos, dithiopyr, thiazopyr, propyzamide, tebutam, chlorthal, carbetamide, chlorbufam, chlorpropham and propham;
   VLCFA inhibitors, such as, for example, acetochlor, alachlor, butachlor, butenachlor, delachlor, diethatyl, dimethachlor, dimethenamid, dimethenamid-P, metazachlor, metolachlor, S-metolachlor, pretilachlor, propachlor, propisochlor, prynachlor, terbuchlor, thenylchlor, xylachlor, allidochlor, CDEA, epronaz, diphenamid, napropamide, naproanilide, pethoxamid, flufenacet, mefenacet, fentrazamide, anilofos, piperophos, cafenstrol, indanofan and tridiphane;
   Cellulose biosynthesis inhibitors, such as, for example, dichlobenil, chlorthiamid, isoxaben and flupoxam;
   Decoupler herbicides, such as, for example, dinofenate, dinoprop, dinosam, dinoseb, dinoterb, DNOC, etinofen and medinoterb;
   Auxine herbicides, such as, for example, clomeprop, 2,4-D, 2,4,5-T, MCPA, MCPA thioethyl, dichlorprop, dichlorprop-P, mecoprop, mecoprop-P, 2,4-DB, MCPB, chloramben, dicamba, 2,3,6-TBA, tricamba, quinclorac, quinmerac, clopyralid, fluoroxypyr, picloram, triclopyr and benazolin;
   Auxine transport inhibitors, such as, for example, naptalam and diflufenzopyr; and benzoylprop, flamprop, flamprop-M, bromobutide, chlorflurenol, cinmethylin, methyldymron, etobenzanid, fosamine, metam, pyributicarb, oxaziclomefone, dazomet, triaziflam and methyl bromide.

Insecticides (a3) comprise, for example, antibiotic insecticides, such as allosamidin, thuringiensin; in particular macrocyclic lactone insecticides, such as spinosad; in particular vermectin insecticides, such as abamectin, doramectin, emamectin, eprinomectin, ivermectin, selamectin; and milbemycin insecticides, such as lepimectin, milbemectin, milbemycin-oxime, moxidectin; arsenic insecticides, such as calcium arsenate, copper acetarsenite, copper arsenate, lead arsenate, potassium arsenite, sodium arsenite; plant-based insecticides, such as anabasin, azadirachtin, D-limonene, nicotin, pyrethrins, cinerin E, cinerin I, cinerin II, jasmolin I, jasmolin II, pyrethrin I, pyrethrin II, quassia, rotenone, ryania, sabadilla; carbamate insecticides, such as bendiocarb, carbaryl; in particular benzofuranyl methylcarbamate insecticides, such as benfuracarb, carbofuran, carbosulfan, decarbofuran, furathiocarb; dimethylcarbamate insecticides, such as dimetan, dimetilan, hyquincarb, pirimicarb; oxime carbamate insecticides, such as alanycarb, aldicarb, aldoxycarb, butocarboxim, butoxycarboxim, methomyl, nitrilacarb, oxamyl, tazimcarb, thiocarboxime, thiodicarb, thiofanox; and phenyl methylcarbamate insecticides, such as allyxycarb, aminocarb, bufencarb, butacarb, carbanolate, cloethocarb, dicresyl, dioxacarb, EMPC, ethiofencarb, fenethacarb, fenobucarb, isoprocarb, methiocarb, metolcarb, mexacarbate, promacyl, promecarb, propoxur, trimethacarb, XMC, xylylcarb; dinitrophenol insecticides, such as dinex, dinoprop, dinosam, DNOC; insect growth regulators; in paritcular chitin synthesis inhibitors, such as bistrifluoron, buprofezin, chlorfluazuron, cyromazine, diflubenzuron, flucycloxuron, flufenoxuron, hexaflumuron, lufenuron, novaluron, noviflumuron, penfluoron, teflubenzuron, triflumuron; juvenile hormone mimetics, such as epofenonane, fenoxycarb, hydroprene, kinoprene, methoprene, pyriproxyfen, triprene; juvenile hormones, such as juvenile hormone I, II and III; molting hormone agonists, such as chromafenozide, halofenozide, methoxyfenozide, tebufenozide; molting hormones, such as α-ecdysone, ecdysterone; molting inhibitors, such as diofenolan; precocenes, such as precocene I, precocene II and precocene III; and unclassified insecticides, such as dicyclanil; nereistoxin analogs, such as bensultap, cartap, thiocyclam, thiosultap; nicotinoid insecticides, such as flonicamid; in particular nitroguanidine insecticides, such as clothianidin, dinotefuran, imidacloprid, thiamethoxam; nitromethylene insecticides, such as nitenpyram, nithiazine; and pyridylmethylamine insecticides, such as acetamiprid, imidacloprid, nitenpyram, thiacloprid; organochlorine insecticides, such as isobenzan, isodrin, kelevan, mirex; organophosphorus insecticides; in particular organophosphate insecticides, such as bromfenvinfos, chlorfenvinphos, crotoxyphos, dichlorvos, dicrotophos, dimethylvinphos, fospirate, heptenophos, methocrotophos, mevinphos, monocrotophos, naled, naftalofos, phosphamidon, propaphos, TEPP, tetrachlorvinphos; organothiophosphate insecticides, such as dioxabenzofos, fosmethilan, phenthoate; in particular aliphatic organothiophosphate insecticides, such as acethion, amiton, cadusafos, chlorethoxyfos, chlormephos, demephion, demephion-O, demephion-S, demeton, demeton-O, demeton-S, demeton-methyl, demeton-O-methyl, demeton-S-methyl, demeton-S-methylsulphon, disulfoton, ethion, ethoprophos, IPSP, isothioate, malathion, methacrifos, oxydemeton-methyl, oxydeprofos, oxydisulfoton, phorate, sulfotep, terbufos, thiometon; in particular aliphatic amideorganothiophosphate insecticides, such as amidithion, cyanthoate, dimethoate, ethoate-methyl, formothion, mecarbam, omethoate, prothoate, sophamide, vamidothion; and oximeorganothiophosphate insecticides, such as chlorphoxim, phoxim, phoxim-methyl; heterocyclic organothiophosphate insecticides, such as azamethiphos, coumaphos, coumithoate, dioxathion, endothion, menazon, morphothion, phosalon, pyraclofos, pyridaphenthion, quinothion; especially benzothiopyran-organothiophosphate insecticides, such as dithicrofos, thicrofos; benzotriazine organothiophosphate insecticides, such as azinphos-ethyl, azinphos-methyl; isoindole organothiophosphate insecticides, such as dialifos, phosmet; isoxazole organothiophosphate insecticides, such as isoxathion, zolaprofos; pyrazolopyrimidine organothiophosphate insecticides, such as chlorprazophos, pyrazophos; pyridine organothiophosphate insecticides, such as chlorpyrifos, chlorpyrifos-methyl; pyrimidine organothiophosphate insecticides, such as butathiofos, diazinon, etrimfos, lirimfos, pirimiphos-ethyl, pirimiphos-methyl, primidophos, pyrimitate, tebupirimfos; quinoxaline organothiophosphate insecticides, such as quinalphos, quinalphos-methyl; thiadiazole organothiophosphate insecticides, such as athidathion, lythidathion, methidathion, prothidathion; and triazole organothiophosphate insecticides, such as isazofos, triazophos; and phenyl organothiophosphate insecticides, such as azothoate, bromophos, bromophos-ethyl, carbophenothion, chlorthiophos, cyanophos, cythioate, dicapthon, dichlofenthion, etaphos, famphur, fenchiorphos, fenitrothion, fensulfothion, fenthion, fenthion-ethyl, heterophos, jodfenphos, mesulfenfos, parathion, parathion-methyl, phenkapton, phosnichlor, profenofos, prothiofos, sulprofos, temephos, trichlormetaphos-3, trifenofos; phosphonate insecticides, such as butonate, trichlorfon; phosphonothioate-insecticides, such as mecarphon; in particular phenyl ethylphosphonothioate-insecticides, such as fonofos, trichloronat; and phenyl phenylphosphonothioate insecticides, such as cyanofenphos, EPN, leptophos; phosphoramidate insecticides, such as crufomate, fenamiphos, fosthietan, mephosfolan, phosfolan, pirimetaphos; phosphoramidothioate insecticides, such as acephate, isocarbophos, isofenphos, methamidophos, propetamphos; and phosphorodiamide insecticides, such as dimefox, mazidox, mipafox, schradan; oxadiazine insecticides, such as indoxacarb; phthalimide insecticides, such as dialifos, phosmet, tetramethrin; pyrazole insecticides, such as acetoprol, ethiprol, fipronil, pyrafluprol, pyriprol, tebufenpyrad, tolfenpyrad, vaniliprole; pyrethroid insecticides; in particular pyrethroid ester insecticides, such as acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, taufluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin; and pyrethroid ether insecticides, such as etofenprox, flufenprox, halfenprox, protrifenbute, silafluofen; pyrimidinamine insecticides, such as flufenerim, pyrimidifen; pyrrole insecticzides, such as chlorfenapyr; tetronic acid insecticides, such as spiromesifen; thiourea insecticides, such as diafenthiuron; urea insecticides, such as flucofuron, sulcofuron; unclassified insecticides, such as closantel, crotamiton, EXD, fenazaflor, fenoxacrim, flubendiamide, hydramethylnon, isoprothiolane, malonoben, metaflumizon, metoxadiazon, nifluridide, pyridaben, pyridalyl, rafoxanide, triarathene, triazamate.

In accordance with a particular embodiment of the present invention, insecticides (a3) comprise:

Organophosphates, such as azinphos-methyl, azinphos-ethyl, chlorpyrifos, chlorpyrifos-methyl, chlorfenvinphos, diazinon, dimethylvinphos, dioxabenzofos, disulfoton, ethion, EPN, fenitrothion, fenthion, heptenophos, isoxathion, malathion, methidathion, methyl-parathion, paraoxon, parathion, phenthoate, phosalone, phosmet, phorate, phoxim, pirimiphos-methyl, profenofos, prothiofos, primiphos-ethyl, pyraclofos, pyridaphenthion, sulprofos, triazophos, trichlorfon, tetrachlorvinphos, vamidothion;
Carbamates, such as alanycarb, benfuracarb, bendiocarb, carbaryl, carbofuran, carbosulfan, fenoxycarb, furathiocarb, indoxacarb, methiocarb, pirimicarb, propoxur, thiodicarb, triazamate;
Pyrethroids, such as bifenthrin, cyfluthrin, cycloprothrin, cypermethrin, deltamethrin, esfenvalerate, ethofenprox, fenpropathrin, fenvalerate, cyhalothrin, lambda-cyhalothrin, permethrin, silafluofen, tau-fluvalinate, tefluthrin, tralomethrin, alpha-cypermethrin, permethrin;
Arthropod growth regulators:
Chitin synthese inhibitors, for example benzoylureas, such as chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine;
Ecdyson antagonists, such as halofenozide, methoxyfenozide, tebufenozide;
Juvenoids, such as pyriproxyfen, methoprene;
Lipid biosynthesis inhibitors, such as spirodiclofen;
Neonicotinoids, such as flonicamid, clothianidin, dinotefuran, imidacloprid, thiamethoxam, nithiazine, acetamiprid, thiacloprid;
Other insecticides which do not belong to any of the classes mentioned, such as abamectin, acequinocyl, acetamiprid, azadirachtin, bensultap, bifenazate, cartap, chlorfenapyr, diafenthiuron, dinetofuran, diofenolan, emamectin, ethiprol, fenazaquin, fipronil, hydramethylnon, imidacloprid, indoxacarb, isoprocarb, metolcarb, pyridaben, pymetrozine, spinosad, tebufenpyrad, thiamethoxam, xmc and xylylcarb and N-phenylsemicarbazones as described in EP-A462-456, in particular compounds of the formula (IV)

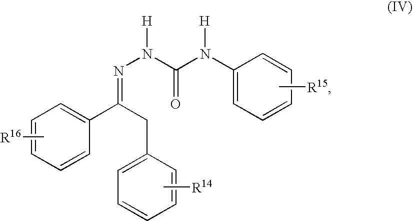

in which R¹⁴ and R¹⁵ independently of one another are hydrogen, halogen, CN, C₁-C₄-alkyl, C₁-C₄-alkoxy, C₁₋₄-haloalkyl or C₁₋₄-haloalkoxy and R¹⁶ is C₁₋₄-alkoxy, C₁₋₄-haloalkyl or C₁₋₄-haloalkoxy, for example compounds according to formula (IV), in which R¹⁶=3-CF₃, R¹⁴=4-CN and R¹⁵=4-OCF₃.

It is also possible to employ salts, in particular agriculturally useful salts, of the active ingredients mentioned specifically in this context.

In a particular embodiment of the invention, the plant protection agent is a fungicide.

It is especially preferred in this context that the fungicide is an active ingredient selected from the group of the anilides, triazolopyrimidines, strobilurins or triazoles, in particular an anilide selected among boscalid, carboxin, metalaxyl and oxadixyl, the triazolopyrimidine 5-chloro-7-(4-methylpiperidin-1-yl)-6-(2,4,6-trifluorophenyl)-[1,2,4]triazol[1,5-a]-pyrimidine, a strobilurin selected among azoxystrobin, pyraclostrobin, dimoxystrobin, trifloxystrobin, fluoxystrobin, picoxystrobin and orysastrobin, or a triazole selected among epoxiconazole, metconazole, tebuconazole, flusilazol, fluquinconazole, triticonazole, propiconazole, penconazole, cyproconazole and prothioconazole.

Epoxiconazole is especially preferred in accordance with the invention.

The names chosen here, of plant protection agents, for example epoxiconazole, include isomeric forms of this compound. Stereoisomers, such as enantiomers or diastereoisomers of the formulae, must be mentioned in particular. In addition to the essentially pure isomers, the compounds of the formulae also include their isomer mixtures, for example stereoisomer mixtures.

Active ingredients with a higher content of the stereoisomer which is biologically more active than the optical antipode, especially preferably isomerically pure active ingredients, are generally preferred.

The present invention particularly relates to compositions with high active-ingredient contents (concentrates). Thus, component (a) will, as a rule, amount to more than 5% by weight, preferably more than 10% by weight and in particular more than 20% by weight of the total weight of the composition. On the other hand, component (a) will expediently amount, as a rule, to less than 80% by weight, preferably less than 70% by weight and in particular less than 60% by weight of the total weight of the composition.

In order to ensure sufficient adjuvant effect, the weight ratio of component (b) to component (a) is preferably more than 0.5, in particular more than 1 and advantageously more than 2.

Furthermore, the compositions according to the invention may comprise auxiliaries and/or additives which are customary for the preparation of formulations in the agrochemical sector, and in particular in the crop protection sector. These include, for example, surfactants, dispersants, wetters, thickeners, organic solvents, cosolvents, antifoams, carboxylic acids, preservatives, stabilizers and the like.

In accordance with a particular embodiment of the present invention, the compositions comprise, as surface-active component (c), at least one (further) surfactant. In this context, the term “surfactant” refers to interface-active or surface-active agents.

Component (c) is added in particular as a dispersant or emulsifier, mainly to disperse a solid component in suspension concentrates. Component (c) may furthermore act in part as wetter.

Substances which are useful in principle are anionic, cationic, amphoteric and nonionic surfactants, with polymer surfactants and surfactants with heteroatoms in the hydrophobic group being included.

The anionic surfactants include, for example, carboxylates, in particular alkali metal, alkaline earth metal and ammonium salts of fatty acids, for example potassium stearate, which are usually also referred to as soaps; acyl glutamates; sarcosinates, for example sodium lauroyl sarcosinate; taurates; methylcelluloses; alkyl phosphates; in particular alkyl esters of mono- and diphosphoric acid; sulfates, in particular alkyl sulfates and alkyl ether sulfates; sulfonates, furthermore alkyl- and alkylarylsulfonates; in particular alkali metal, alkaline earth metal and ammonium salts of arylsulfonic acids, and alkyl-substituted arylsulfonic acids, alkylbenzenesulfonic acids, such as, for example, lignosulfonic and phenolsulfonic acid, naphthalene- and dibutylnaphthalenesulfonic acids, or dodecylbenzenesulfonates, alkylnaphthalenesulfonates, alkyl methyl estersulfonates, condensates of sulfonated naphthalene and derivatives thereof with formaldehyde, condensates of naphthalenesulfonic acids, phenolic and/or phenolsulfonic acids with formaldehyde or with formaldehyde and urea, mono- or dialkylsuccinicestersulfonates; and protein hydrolyzates and lignin-sulfite waste liquors. The abovementioned sulfonic acids are advantageously used in the form of their neutral or, optionally, basic salts.

The cationic surfactants include, for example, quaternized ammonium compounds, in particular alkyltrimethylammonium and dialkyldimethylammonium halides and alkyltrimethylammonium and dialkyldimethylammonium alkyl sulfates, and pyridine and imidazoline derivatives, in particular alkylpyridinium halides.

The nonionic surfactants include, for example, further alkoxylates and especially ethoxylates, and nonionic surfactants, in particular
fatty alcohol polyoxyethylene esters, for example lauryl alcohol polyoxyethylene ether acetate;
alkyl polyoxyethylene ethers and alkyl polyoxypropylene ethers, for example of fatty alcohols having 8 or more carbon atoms,
alkylaryl alcohol polyoxyethylene ethers, for example octylphenol polyoxyethylene ether,
alkoxylated animal and/or vegetable fats and/or oils, for example, corn oil ethoxylates, castor oil ethoxylates, tallow fat ethoxylates,
glycerol esters, such as, for example, glycerol monostearate,
alkylphenol alkoxylates, such as, for example, ethoxylated iso-octylphenol, octylphenol or nonylphenol, tributylphenol polyoxyethylene ether,
fatty amine alkoxylates, fatty acid amide alkoxylates and fatty acid diethanolamide alkoxylates, in particular their ethoxylates,
sugar surfactants, sorbitol esters, such as, for example sorbitan fatty acid esters (sorbitan monooleate, sorbitan tristearate), polyoxyethylene sorbitan fatty acid esters, alkyl polyglycosides, N-alkylgluconamides,
alkylmethylsulfoxides,
alkyldimethylphosphine oxides, such as, for example, tetradecyldimethyl-phosphine oxide.

The amphoteric surfactants include, for example, sulfobetains, carboxybetains and alkyldimethylamine oxides, for example tetradecyldimethylamine oxide.

The polymeric surfactants include, for example, di-, tri-, and multiblock polymers of the type (AB)ₓ, ABA and BAB, e.g. if appropriate end-group-closed ethylene-oxide/propylene oxide block copolymers, e.g. ethylene diamine/EO/PO block copolymers, polystyrene/block/polyethylene oxide, and AB comb polymers, e.g. polymethacrylate/comb/polyethylene oxide.

Further surfactants to be mentioned by way of example in this context are perfluorine surfactants, silicone surfactants, for example polyether-modified siloxanes, phospholipids, such as, for example, lecithin or chemically modified lecithins, amino acid surfactants, for example N-lauroylglutamate, and surface-active homo- and copolymers, for example polyvinylpyrrolidone, polyacrylic acids in the form of their salts, polyvinyl alcohol, polypropylene oxide, polyethylene oxide, maleic anhydride/isobutene copolymers and vinylpyrrolidone/vinyl acetate copolymers.

Unless specified, the alkyl chains of the abovementioned surfactants are linear or branched radicals having, usually, 8 to 20 carbon atoms.

The further surfactant within the scope of component (c) is preferably selected among nonionic surfactants. Among these, surfactants with HLB values in the range from 2 to 16, preferably in the range from 5 to 16 and in particular in the range of from 8 to 16, are preferred.

Component (c)—if present—will, as a rule, amount to less than 50% by weight, preferably less than 15% by weight and in particular less than 5% by weight of the total weight of the composition.

In accordance with a particular embodiment of the present invention, the compositions comprise, as component (d), at least one further auxiliary.

Component (d) may serve a multitude of purposes. The skilled worker will choose suitable auxiliaries in the customary manner to meet the specific requirements.

For example, further auxiliaries are selected among

(d1) solvents or diluents;
(d2) retention agents, pH buffers, antifoams.

Besides water, the compositions may comprise further solvents of soluble constituents, or diluents for insoluble constituents of the composition.

Substances which are useful in principle are, for example, mineral oils, synthetic oils and vegetable and animal oils, and low-molecular-weight hydrophilic solvents such as alcohols, ethers, ketones and the like.

Substances which must therefore be mentioned are, firstly, aprotic or apolar solvents or diluents, such as mineral oil fractions of medium to high boiling point, for example kerosene and diesel oil, furthermore coal tar oils, hydrocarbons, liquid paraffins, for example C₈- to C₃₀-hydrocarbons of the n- or iso-alkane series or mixtures of these, optionally, hydrogenated or partially hydrogenated aromatics or alkyl aromatics from the benzene or naphthalene series, for example aromatic or cycloaliphatic C₇- to C₁₈-hydrocarbon compounds, aliphatic or aromatic carboxylic acid or dicarboxylic acid esters, fats or oils of vegetable or animal origin, such as mono-, di- and triglycerides, in pure form or as a mixture, for example in the form of oily extracts from natural substances, for example olive oil, soy oil, sunflower oil, castor oil, sesame oil, corn oil, peanut oil, rapeseed oil, linseed oil, almond oil, castor oil, safflower oil, and their raffinates, for example hydrogenated or partially hydrogenated products thereof and/or their esters, in particular methyl and ethyl esters.

Examples of C₈- to C₃₀-hydrocarbons of the n- or iso-alkane series are n- and iso-octane, -decane, -hexadecane, -octadecane, -eicosane, and preferably hydrocarbon mixtures, such as liquid paraffin (which, if industrial-grade, may comprise up to approximately 5% aromatics) and a C₁₈-C₂₄-mixture which is commercially obtainable from Texaco under the name Spraytex oil.

The aromatic or cycloaliphatic C₇- to C₁₈-hydrocarbon compounds include, in particular, aromatic or cycloaliphatic solvents from the series of the alkyl aromatics. These compounds may be unhydrogenated, partially hydrogenated or fully hydrogenated. Such solvents include, in particular, mono-, di- or trialkylbenzenes, mono-, di-, trialkyl-substituted tetralins and/or mono-, di-, tri- or tetraalkyl-substituted naphthalenes (alkyl preferably represents C₁-C₆-alkyl). Examples of such solvents are toluene, o-, m-, p-xylene, ethylbenzene, isopropylbenzene, tert-butylbenzene and mixtures, such as the products from Exxon sold under the names Shelisol and Solvesso, for example Solvesso 100, 150 and 200.

Examples of suitable monocarboxylic acid esters are oleic esters, in particular methyl oleate and ethyl oleate, lauric acid esters, in particular 2-ethylhexyl laurate, octyl laurate and isopropyl laurate, isopropyl myristate, palmitic acid esters, in particular 2-ethylhexyl palmitate and isopropyl palmitate, stearic acid esters, in particular n-butyl stearate and 2-ethylhexyl 2-ethylhexanoate.

Examples of suitable dicarboxylic acid esters are adipic acid esters, in particular dimethyl adipate, di-n-butyl adipate, di-n-octyl adipate, di-iso-octyl adipate, also referred as bis-(2-ethylhexyl) adipate, di-n-nonyl adipate, di-iso-nonyl adipate and ditridecyl adipate; succinic acid esters, in particular di-n-octyl succinate and di-iso-octyl succinate, and di-(iso-nonyl)cyclohexane-1,2-dicarboxylate.

As a rule, the above-described aprotic solvents or diluents amount to less than 80%, preferably less than 50% and in particular less than 30% of the total weight of the composition.

Some of these aprotic solvents or diluents can also have adjuvant properties, i.e. in particular activity-enhancing properties. This applies in particular to said mono- and dicarboxylic acid esters. In accordance with this aspect, such adjuvants may also, as part of another formulation (stand-alone product), be mixed with the alcohol alkoxylates according to the invention, or with compositions comprising them, at a suitable point in time, as a rule shortly before application.

Secondly, protic or polar solvents or diluents must be mentioned, for example C₂-C₈-mono alcohols, such as ethanol, propanol, isopropanol, butanol, isobutanol, tert-butanol, cyclohexanol and 2-ethylhexanol, C₃-C₈-ketones, such as diethyl ketone, t-butyl methyl ketone, cyclohexanone and 2-sec-butylphenol, and aprotic amines, such as N-methyl- and N-octylpyrrolidone.

As a rule, the above-described protic or polar solvents or diluents amount to less than 80%, preferably less than 50% and in particular less than 30% of the total weight of the composition.

It is also possible to use antisettling agents, in particular for suspension concentrates. Such antisettling agents serve in particular the purpose of rheological stabilization. Substances which must be mentioned in this context are, in particular, mineral products, for examples bentonites, talcites and hectorites.

Other additives which may optionally be useful can be found for example among mineral salt solutions, which are employed for alleviating nutritional and trace element deficiencies, nonphytotoxic oils and oil concentrates, antidrift reagents, antifoams, in particular those of the silicone type, for example Silicon SL, which is commercially obtainable from Wacker, and the like.

In accordance with a preferred embodiment, the compositions according to the invention are liquid formulations.

The formulations may be present for example as emulsifiable concentrate (EC), suspoemulsion (SE), oil-in-water emulsion (0/W), water-in-oil emulsion (W/O), aqueous suspension concentrate, oil suspension concentrate (OD), microemulsion (ME).

The compositions can be prepared in a manner known per se. To this end, at least some of the components are combined. It must be noted that products, in particular commercially available products, can be used whose constituents may contribute to different components. For example, a specific surfactant may be dissolved in an aprotic solvent, so that this product may contribute to various components. Furthermore, it is also possible, under certain circumstances, that small amounts of less desired substances are introduced together with commercially available products. As a mixture, the combined products must then, as a rule, be mixed finely with one another to give a homogeneous mixture and, if required, ground, for example in the case of suspensions.

Mixing can be effected in a manner known per se, for example by homogenizing with suitable devices such as KPG or magnetic stirrers. Grinding, too, is a process which is known per se. Grinding media which may be employed are grinding media made of glass, or other mineral or metallic grinding media, as a rule in a size of from 0.1-30 mm and in particular of from 0.6-2 mm. As a rule, the mixture is ground until the desired particle size has been reached.

Before use, the compositions are converted into a suitable application form in the customary manner, as a rule by dilution. Dilution with water or else aprotic solvents, for example in the tank mix method, is preferred. The use in the form of a spray mixture preparation is preferred. It may be applied pre- or post-emergence. Post-emergence application leads to particular advantages.

The use according to the invention also comprises the use of the alkoxylates according to the invention as stand-alone product. To this end, the alkoxylates are prepared in a suitable manner in order to be added, shortly before application, to the product to be applied. As regards the ratio between alkoxylate and active ingredient, what has been said above in connection with the composition also applies here. In this sense, the combination according to the invention of active ingredient and adjuvant may also be provided in the form of a kit. Such a kit comprises at least two containers. One container comprises at least one active ingredient for the treatment of plants, if appropriate formulated as a composition together with expedient auxiliaries. A further container comprises at least one alcohol alkoxylate of the formula (I).

Particular advantages result especially in the case of a spray application. For a conventional tank-mix spray mixture, the compositions, which already comprise an alkoxylated branched alcohol,—or further plant treatment compositions with the addition of at least one alkoxylated branched alcohol as stand-alone product—are diluted with water in such a way that approximately 0.01 to 10, preferably approximately 0.05 to 5 and in particular 0.1 to 1 kg of at least one alkoxylate according to the invention are applied per ha.

In the context of the present description, quantities generally refer to the total weight of a composition, unless otherwise specified. In accordance with the invention, the term “essentially” generally relates to a percentage ratio of at least 80% or preferably at least 90% and in particular at least 95%.

The invention is illustrated in greater detail by the examples which follow:

The weight-average molecular weights of the alkoxylates according to the invention can be determined by gel permeation chromatography as specified in DIN 55672.

Protocol for determining the iso-index of an alcohol mixture of secondary and/or primary alcohols via ¹H NMR:

Approximately 20 mg of alcohol mixture are dissolved in 0.4 ml of CDCl₃, and a small amount of TMS is added as the frequency reference. Thereafter, the solution is treated with 0.2 ml of TAI, charged into a 5 mm NMR tube and measured in the NMR spectrometer.

Measuring conditions:
spectrometer frequency: 400 MHZ
relaxation delay: 10 s
pulse angle: 30°
data points recorded: 64 K
scan number: 64
transformed data points 64 K
exponential multiplication: 0.2 Hz

Following Fourier transformation, automatic phase and baseline correction, the ranges 5.4 to 3.7 ppm (all TAI-esterified secondary or primary alcohols) and 2.4 to 0.4 ppm (all methyl, methylene and methyne protons) are manually integrated. Here, the zero-order integral phases are selected in such a way that the beginning and the end of the integral curves are essentially horizontal. The signals<1 ppm are assigned to the methyl groups.

### PREPARATION EXAMPLES

### Reference Examples 1 to 24

### Preparation of the Alkoxylates (a) to (x)

### Reference Example 1

### 1-Heptanol+17 PO+5 EO (a)

A rotary evaporator was initially charged with 116.2 g of 1-heptanol (corresponding to 1.0 mol) together with 5.3 g of 50% strength potassium hydroxide (alkoxylation catalyst; corresponding to 0.2% by weight based on the total mixture), and the reaction mixture was dehydrated and transferred into an autoclave.

At 130° C., initially 986 g of propylene oxide (corresponding to 17.0 mol) and then 220.0 g of ethylene oxide (corresponding to 5.0 mol) were passed in continuously.

To complete the conversion, stirring of the mixture was continued for 30 minutes while simultaneously cooling to 80° C.

This gave 1336 g of the alcohol alkoxylate (a).

### Reference Example 2

### Dipropylene Glycol Monomethyl Ether+20 PO+1 EO (b)

An autoclave was initially charged with 19.4 g of dipropylene glycol monomethyl ether (corresponding to 0.12 mol) together with 0.66 g of 50% strength potassium hydroxide (alkoxylation catalyst; corresponding to 0.2% by weight based on the total mixture). The mixture was dehydrated in the autoclave.

First, 139.2 g of propylene oxide (corresponding to 2.4 mol) and then 5.3 g of ethylene oxide (corresponding to 0.12 mol) were passed in continuously at 130° C.

To complete the conversion, stirring of the mixture was continued for 30 minutes while simultaneously cooling to 80° C.

This gave 162.0 g of the alcohol alkoxylate (b).

### Reference Example 3

### Dipropylene Glycol Monobutyl Ether+1 PO+10 EO (c)

An autoclave was initially charged with 47.5 g of dipropylene glycol monobutyl ether (corresponding to 0.25 mol) together with 0.69 g of 50% strength potassium hydroxide (alkoxylation catalyst; corresponding to 0.2% by weight based on the total mixture). The mixture was dehydrated in the autoclave.

First, 14.5 g of propylene oxide (corresponding to 0.25 mol) and then 110.0 g of ethylene oxide (corresponding to 2.5 mol) were passed in continuously at 130° C.

To complete the conversion, stirring of the mixture was continued for 30 minutes while simultaneously cooling to 80° C.

This gave 158.0 g of the alcohol alkoxylate (c).

### Reference Example 4

### Dipropylene Glycol Monopropyl Ether+20 PO+10 EO (d)

An autoclave was initially charged with 21.1 g of dipropylene glycol monopropyl ether (corresponding to 0.12 mol) together with 0.85 g of 50% strength potassium hydroxide (alkoxylation catalyst; corresponding to 0.2% by weight based on the total mixture). The mixture was dehydrated in the autoclave.

First, 139.2 g of propylene oxide (corresponding to 2.4 mol) and then 52.8 g of ethylene oxide (corresponding to 1.2 mol) were passed in continuously at 130° C.

To complete the conversion, stirring of the mixture was continued for 30 minutes while simultaneously cooling to 80° C.

This gave 196.0 g of the alcohol alkoxylate (d).

### Reference Example 5

### Dipropylene Glycol Monobutyl Ether+10.5 PO+5.5 EO (e)

An autoclave was initially charged with 28.5 g of dipropylene glycol monobutyl ether (corresponding to 0.15 mol) together with 0.62 g of 50% strength potassium hydroxide (alkoxylation catalyst; corresponding to 0.2% by weight based on the total mixture). The mixture was dehydrated in the autoclave.

First, 91.4 g of propylene oxide (corresponding to 1.6 mol) and then 36.3 g of ethylene oxide (corresponding to 0.83 mol) were passed in continuously at 130° C.

To complete the conversion, stirring of the mixture was continued for 30 minutes while simultaneously cooling to 80° C.

This gave 145.0 g of the alcohol alkoxylate (e).

### Reference Example 6

### 1-Hexanol+10.5 PO+5.5 EO (f)

An autoclave was initially charged with 20.4 g of 1-hexanol (corresponding to 0.2 mol) together with 0.38 g of potassium tert-butoxide (alkoxylation catalyst; corresponding to 0.2% by weight based on the total mixture).

First, 121.8 g of propylene oxide (corresponding to 2.1 mol) and then 48.4 g of ethylene oxide (corresponding to 1.1 mol) were passed in continuously at 130° C.

To complete the conversion, stirring of the mixture was continued for 30 minutes while simultaneously cooling to 80° C.

This gave 189.0 g of the alcohol alkoxylate (f).

### Reference Example 7

### 1-Heptanol+3 BO+5 EO (g)

An autoclave was initially charged with 116.2 g of 1-heptanol (corresponding to 1.0 mol) together with 2.76 g of potassium tert-butoxide (alkoxylation catalyst; corresponding to 0.5% by weight based on the total mixture).

First, 216.0 g of 1,2-butylene oxide (corresponding to 3.0 mol) and then 220.0 g of ethylene oxide (corresponding to 5.0 mol) were passed in at 130° C.

To complete the conversion, stirring of the mixture was continued for 30 minutes while simultaneously cooling to 80° C.

This gave 553.3 g of the alcohol alkoxylate (g).

The weight-average molecular weight determined by means of gel permeation chromatography as specified in DIN 55672 was approximately 550.

### Reference Example 8

### 1-Heptanol+5 BO+5 EO (h)

An autoclave was initially charged with 116.2 g of 1-heptanol (corresponding to 1.0 mol) together with 3.48 g of potassium tert-butoxide (alkoxylation catalyst; corresponding to 0.5% by weight based on the total mixture).

First, 360.0 g of 1,2-butylene oxide (corresponding to 5.0 mol) and then 220.0 g of ethylene oxide (corresponding to 5.0 mol) were passed in at 130° C.

To complete the conversion, stirring of the mixture was continued for 30 minutes while simultaneously cooling to 80° C.

This gave 727.3 g of the alcohol alkoxylate (h).

The weight-average molecular weight determined by means of gel permeation chromatography as specified in DIN 55672 was approximately 725.

### Reference Example 9

### 1-Heptanol+7 BO+5 EO (i)

An autoclave was initially charged with 116.2 g of 1-heptanol (corresponding to 1.0 mol) together with 4.2 g of potassium tert-butoxide (alkoxylation catalyst; corresponding to 0.5% by weight based on the total mixture).

First, 504.0 g of 1,2-butylene oxide (corresponding to 7.0 mol) and then 220.0 g of ethylene oxide (corresponding to 5.0 mol) were passed in at 130° C.

To complete the conversion, stirring of the mixture was continued for 30 minutes while simultaneously cooling to 80° C.

This gave 889.5 g of the alcohol alkoxylate (i).

The weight-average molecular weight determined by means of gel permeation chromatography as specified in DIN 55672 was approximately 840.

### Reference Example 10

### 1-Heptanol+3 BO+12 EO (j)

An autoclave was initially charged with 116.2 g of 1-heptanol (corresponding to 1.0 mol) together with 1.72 g of potassium tert-butoxide (alkoxylation catalyst; corresponding to 0.2% by weight based on the total mixture).

First, 216.0 g of 1,2-butylene oxide (corresponding to 3.0 mol) and then 528.0 g of ethylene oxide (corresponding to 12.0 mol) were passed in at 130° C.

To complete the conversion, stirring of the mixture was continued for 30 minutes while simultaneously cooling to 80° C.

This gave 844 g of the alcohol alkoxylate (j).

The weight-average molecular weight determined by means of gel permeation chromatography as specified in DIN 55672 was approximately 860.

### Reference Example 11

### 1-Heptanol+9 BO+5 EO (k)

An autoclave was initially charged with 116.2 g of 1-heptanol (corresponding to 1.0 mol) together with 1.97 g of potassium tert-butoxide (alkoxylation catalyst; corresponding to 0.2% by weight based on the total mixture).

First, 648.0 g of 1,2-butylene oxide (corresponding to 9.0 mol) and then 220.0 g of ethylene oxide (corresponding to 5.0 mol) were passed in at 130° C.

To complete the conversion, stirring of the mixture was continued for 30 minutes while simultaneously cooling to 80° C.

This gave 970 g of the alcohol alkoxylate (k).

The weight-average molecular weight determined by means of gel permeation chromatography as specified in DIN 55672 was approximately 980.

### Reference Example 12

### 1-Heptanol+9 BO+12 EO (l)

An autoclave was initially charged with 116.2 g of 1-heptanol (corresponding to 1.0 mol) together with 2.58 g of potassium tert-butoxide (alkoxylation catalyst; corresponding to 0.2% by weight based on the total mixture).

First, 648.0 g of 1,2-butylene oxide (corresponding to 9.0 mol) and then 528.0 g of ethylene oxide (corresponding to 12.0 mol) were passed in at 130° C.

To complete the conversion, stirring of the mixture was continued for 30 minutes while simultaneously cooling to 80° C.

This gave 1325 g of the alcohol alkoxylate (I).

The weight-average molecular weight determined by means of gel permeation chromatography as specified in DIN 55672 was approximately 1290.

### Reference Example 13

### 1-Heptanol+12 EO+9 BO (m)

An autoclave was initially charged with 116.2 g of 1-heptanol (corresponding to 1.0 mol) together with 1.97 g of potassium tert-butoxide (alkoxylation catalyst; corresponding to 0.2% by weight based on the total mixture).

First, 528.0 g of ethylene oxide (corresponding to 12.0 mol) and then 648.0 g of 1,2-butylene oxide (corresponding to 9.0 mol) were passed in at 130° C.

To complete the conversion, stirring of the mixture was continued for 30 minutes while simultaneously cooling to 80° C.

This gave 1260 g of the alcohol alkoxylate (m).

The weight-average molecular weight determined by means of gel permeation chromatography as specified in DIN 55672 was approximately 1290.

### Reference Example 14

### 1-Heptanol+3 BO+12 EO+1 i-BO (n)

An autoclave was initially charged with 174.3 g of 1-heptanol (corresponding to 1.5 mol) together with 2.58 g of potassium tert-butoxide (alkoxylation catalyst; corresponding to 0.2% by weight based on the total mixture).

First, 324.0 g of 1,2-butylene oxide (corresponding to 3.0 mol) and then 792.0 g of ethylene oxide (corresponding to 12.0 mol) were passed in at 130° C.

To complete the conversion, stirring of the mixture was continued for 30 minutes while simultaneously cooling to 80° C.

An autoclave was initially charged with an aliquot 403.0 g of the resulting alcohol alkoxylate (corresponding to 0.5 mol) together with 0.89 g of potassium tert-butoxide (alkoxylation catalyst; corresponding to 0.2% by weight based on the total mixture).

At 140° C., 43.2 g of iso-butylene oxide (corresponding to 0.6 mol) were passed in.

To complete the conversion, stirring of the mixture was continued for 30 minutes while simultaneously cooling to 80° C.

This gave 438 g of the alcohol alkoxylate (n).

The weight-average molecular weight determined by means of gel permeation chromatography as specified in DIN 55672 was approximately 880.

### Reference Example 15

### 1-Heptanol+17 PO+5 EO+1 i-BO (o)

An autoclave was initially charged with 116.2 g of 1-heptanol (corresponding to 1.0 mol) together with 2.65 g of potassium tert-butoxide (alkoxylation catalyst; corresponding to 0.2% by weight based on the total mixture).

First, 986.0 g of 1,2-propylene oxide (corresponding to 17.0 mol) and then 220.0 g of ethylene oxide (corresponding to 5.0 mol) were passed in at 130° C.

To complete the conversion, stirring of the mixture was continued for 30 minutes while simultaneously cooling to 80° C.

An autoclave was initially charged with an aliquot of 462.8 g of the alcohol alkoxylate obtained (corresponding to 0.4 mol) together with 1.0 g of potassium tert-butoxide (alkoxylation catalyst; corresponding to 0.2% by weight based on the total mixture).

34.6 g of iso-butylene oxide (corresponding to 0.48 mol) were passed in at 140° C.

To complete the conversion, stirring of the mixture was continued for 30 minutes while simultaneously cooling to 80° C.

This gave 487 g of the alcohol alkoxylate (o).

The weight-average molecular weight determined by means of gel permeation chromatography as specified in DIN 55672 was approximately 1230.

### Reference Example 16

### 1-Heptanol+3 BO+12 EO+DMS (p)

An autoclave was charged with 174.3 g of 1-heptanol (corresponding to 1.5 mol) together with 2.58 g of potassium tert-butoxide (alkoxylation catalyst; corresponding to 0.2% by weight based on the total mixture).

First, 324.0 g of 1,2-butylene oxide (corresponding to 3.0 mol) and then 792.0 g of ethylene oxide (corresponding to 12.0 mol) were passed in at 130° C.

To complete the conversion, stirring of the mixture was continued for 30 minutes while simultaneously cooling to 80° C.

A four-necked flask was charged with an aliquot, 322.4 g (corresponding to 0.4 mol) of the resulting alkoxylate, and 163.2 g of 50% strength sodium hydroxide solution (corresponding to 2.04 mol) were added dropwise with cooling. Thereafter, 65.6 g of dimethyl sulfate (corresponding to 0.52 mol) were metered into the reaction mixture at no more than 40° C.

This gave 334.1 g of the modified alcohol alkoxylate (p).

The weight-average molecular weight determined by means of gel permeation chromatography as specified in DIN 55672 was approximately 880.

### Reference Example 17

### 1-Heptanol+17 PO+5 EO+DMS (q)

An autoclave was initially charged with 116.2 g of 1-heptanol (corresponding to 1.0 mol) together with 2.65 g of potassium tert-butoxide (alkoxylation catalyst; corresponding to 0.2% by weight based on the total mixture).

At first, 986.0 g of 1,2-propylene oxide (corresponding to 17.0 mol) and then 220.0 g of ethylene oxide (corresponding to 5.0 mol) were passed in at 130° C.

To complete the conversion, stirring of the mixture was continued for 30 minutes while simultaneously cooling to 80° C.

A four-necked flask was initially charged with an aliquot of 462.8 g (corresponding to 0.4 mol) of the alkoxylate obtained, and 163.2 g of 50% strength aqueous sodium hydroxide solution (corresponding to 2.04 mol) were added dropwise with cooling. At a temperature of at most 40° C., 65.6 g of dimethyl sulfate (corresponding to 0.52 mol) were then metered into the reaction mixture.

This gave 451 g of the modified alcohol alkoxylate (q).

The weight-average molecular weight determined by means of gel permeation chromatography as specified in DIN 55672 was approximately 1340.

### Reference Example 18

### Dipropylene Glycol Monobutyl Ether+3 BO+12 EO (r)

An autoclave was initially charged with 190.0 g of dipropylene glycol monobutyl ether (corresponding to 1.0 mol) together with 3.72 g of 50% strength aqueous potassium hydroxide solution (alkoxylation catalyst; corresponding to 0.2% by weight based on the total mixture).

First, 216.0 g of 1,2-butylene oxide (corresponding to 3.0 mol) and then 528.0 g of ethylene oxide (corresponding to 12.0 mol) were passed in at 130° C.

To complete the conversion, stirring of the mixture was continued for 30 minutes while simultaneously cooling to 80° C.

This gave 930 g of the alcohol alkoxylate (r).

The weight-average molecular weight determined by means of gel permeation chromatography as specified in DIN 55672 was approximately 930.

### Reference Example 19

### Dipropylene Glycol Monobutyl Ether+1 BO+12 EO (s)

An autoclave was initially charged with 190.0 g of dipropylene glycol monobutyl ether (corresponding to 1.0 mol) together with 3.16 g of 50% strength aqueous potassium hydroxide solution (alkoxylation catalyst; corresponding to 0.2% by weight based on the total mixture).

First, 72.0 g of 1,2-butylene oxide (corresponding to 1.0 mol) and then 528.0 g of ethylene oxide (corresponding to 12.0 mol) were passed in at 130° C.

To complete the conversion, stirring of the mixture was continued for 30 minutes while simultaneously cooling to 80° C.

This gave 789 g of the alcohol alkoxylate (s).

The weight-average molecular weight determined by means of gel permeation chromatography as specified in DIN 55672 was approximately 790.

### Reference Example 20

### Dipropylene Glycol Monobutyl Ether+1 PeO+12 EO (t)

An autoclave was initially charged with 190.0 g of dipropylene glycol monobutyl ether (corresponding to 1.0 mol) together with 1.61 g of 50% strength aqueous potassium hydroxide solution (alkoxylation catalyst; corresponding to 0.2% by weight based on the total mixture).

First, 86.0 g of 1,2-pentene oxide (corresponding to 1.0 mol) were passed in at 150° C., and then 528.0 g of ethylene oxide (corresponding to 12.0 mol) were passed in at 130° C.

To complete the conversion, stirring of the mixture was continued for 30 minutes while simultaneously cooling to 80° C.

This gave 789 g of the alcohol alkoxylate (t).

The weight-average molecular weight determined by means of gel permeation chromatography as specified in DIN 55672 was approximately 804.

### Reference Example 21

### Dipropylene Glycol Monobutyl Ether+1 DeO+12 EO (u)

An autoclave was initially charged with 190.0 g of dipropylene glycol monobutyl ether (corresponding to 1.0 mol) together with 1.57 g of 50% strength aqueous potassium hydroxide solution (alkoxylation catalyst; corresponding to 0.2% by weight based on the total mixture).

First, 156.0 g of 1,2-decene oxide (corresponding to 1.0 mol) were passed in at 150° C., and then 528.0 g of ethylene oxide (corresponding to 12.0 mol) were passed in at 130° C.

To complete the conversion, stirring of the mixture was continued for 30 minutes while simultaneously cooling to 80° C.

This gave 866 g of the alcohol alkoxylate (u).

The weight-average molecular weight determined by means of gel permeation chromatography as specified in DIN 55672 was approximately 874.

### Reference Example 22

### Dipropylene Glycol Monobutyl Ether+12 EO+1 PeO (v)

An autoclave was initially charged with 190.0 g of dipropylene glycol monobutyl ether (corresponding to 1.0 mol) together with 1.61 g of potassium tert-butoxide (alkoxylation catalyst; corresponding to 0.2% by weight based on the total mixture).

First, 528.0 g of ethylene oxide (corresponding to 12.0 mol) were passed in at 130° C., and then 86.0 g of 1,2-pentene oxide (corresponding to 1.0 mol) were passed in at 150° C.

To complete the conversion, stirring of the mixture was continued for 30 minutes while simultaneously cooling to 80° C.

This gave 815 g of the alcohol alkoxylate (v).

The weight-average molecular weight determined by means of gel permeation chromatography as specified in DIN 55672 was approximately 805.

### Reference Example 23

### Dipropylene Glycol Monobutyl Ether+12 EO+1 DeO (w)

An autoclave was initially charged with 190.0 g of dipropylene glycol monobutyl ether (corresponding to 1.0 mol) together with 1.75 g of potassium tert-butoxide (alkoxylation catalyst; corresponding to 0.2% by weight based on the total mixture).

First, 528.0 g of ethylene oxide (corresponding to 12.0 mol) were passed in at 130° C., and then 156.0 g of 1,2-decene oxide (corresponding to 1.0 mol) were passed in at 150° C.

To complete the conversion, stirring of the mixture was continued for 30 minutes while simultaneously cooling to 80° C.

This gave 895 g of the alcohol alkoxylate (w).

The weight-average molecular weight determined by means of gel permeation chromatography as specified in DIN 55672 was approximately 874.

### Reference Example 24

### Pentanol Mixture+7 BO+12 EO (x)

An autoclave was initially charged with 87.2 g of pentanol mixture (corresponding to 1.0 mol) together with 2.23 g of potassium tert-butoxide (alkoxylation catalyst; corresponding to 0.2% by weight based on the total mixture).

First, 504.0 g of 1,2-butylene oxide (corresponding to 7.0 mol) and then 528.0 g of ethylene oxide (corresponding to 12.0 mol) were passed in at 130° C.

To complete the conversion, stirring of the mixture was continued for 30 minutes while simultaneously cooling to 80° C.

This gave 1116 g of the alcohol alkoxylate (x).

The weight-average molecular weight determined by means of gel permeation chromatography as specified in DIN 55672 was approximately 1120.

### Example 1

### Fungicidal Efficacy

125 g/l epoxiconazole were ground together with in each case 20 g/l dispersant (Atlas G 5000¹, Synperonic A¹) and 50 g/l propylene glycol in aqueous medium in a stirred-ball mill (dyno-mill) until a particle size of 80%<2 μm was reached. To the mixture were added 3 g/l per liter antifoam, for example Rhodorsil 426², 3 g/l thickener, for example Rhodopol 23², and a biocide, for example Acticide MBS³. The respective adjuvant, in aqueous solution or in a solvent, for example Solvesso, was stirred into this mixture, so that the final concentration of the formulation is composed of 62.5 g/l epoxiconazole and 125 g/l adjuvant. ¹Uniquema/Croda²Rhodia³Thor Chemie

### Biotest (Curative Control of Leaf Rust of Wheat):

Leaves of wheat seedlings of the species “Kanzler” which had been grown in pots were dusted, in the two-leaf stage, with spores of leaf rust of wheat “Puccinia recondita” and incubated for 2 days in the greenhouse at high atmospheric humidity. In fully automated spray cabins, the plants were then sprayed with the formulations which comprised the active ingredients and adjuvants specified hereinbelow. The spray mixtures comprised 50 ppm epoxiconazole and 100 ppm adjuvant. The ratio active ingredient to adjuvant was, accordingly, 1:2. After the spray coatings had dried, the plants were returned to the greenhouse and grown at temperatures between 20 and 24° C. and a relative atmospheric humidity of 60 to 90%. After 10 days, the extent of the leaf rust disease was determined visually as disease percent of the total leaf area. 3 pots were evaluated for each combination.

| Adjuvant | Active ingredient | % |
|---|---|---|
| [100 ppm] | [50 ppm] | infection |
| (a) | Epoxiconazole | 0 |
| (b) | Epoxiconazole | 1 |
| (c) | Epoxiconazole | 4 |
| (d) | Epoxiconazole | 0 |
| (e) | Epoxiconazole | 0 |
| (f) | Epoxiconazole | 0 |
| (g) | Epoxiconazole | 3 |
| (h) | Epoxiconazole | 2 |
| (i) | Epoxiconazole | 1 |
| (j) | Epoxiconazole | 3 |
| (k) | Epoxiconazole | 5 |
| (l) | Epoxiconazole | 0 |
| (m) | Epoxiconazole | 4 |
| (n) | Epoxiconazole | 2 |
| (o) | Epoxiconazole | 6 |
| (p) | Epoxiconazole | 2 |
| (q) | Epoxiconazole | 0 |
| (r) | Epoxiconazole | 3 |
| (s) | Epoxiconazole | 7 |
| (t) | Epoxiconazole | 5 |
| (u) | Epoxiconazole | 3 |
| (v) | Epoxiconazole | 3 |
| (w) | Epoxiconazole | 1 |
| (x) | Epoxiconazole | 0 |
| LeoFAT | Epoxiconazole | 7 |
| — | Epoxiconazole | 40 |
| — | — | 90 |

| | |
|---|---|
| (a) | 1-Heptanol + 17 PO + 5 EO |
| (b) | Dipropylene glycol monomethyl ether + 20 PO + 1 EO |
| (c) | Dipropylene glycol monobutyl ether + 1 PO + 10 EO |
| (d) | Dipropylene glycol monopropyl ether + 20 PO + 10 EO |
| (e) | Dipropylene glycol monobutyl ether + 10.5 PO + 5.5 EO |
| (f) | 1-Hexanol + 10.5 PO + 5.5 EO |
| (g) | 1-Heptanol + 3 BO + 5 EO |
| (h) | 1-Heptanol + 5 BO + 5 EO |
| (i) | 1-Heptanol + 7 BO + 5 EO |
| (j) | 1-Heptanol + 3 BO + 12 EO |
| (k) | 1-Heptanol + 9 BO + 5 EO |
| (l) | 1-Heptanol + 9 BO + 12 EO |
| (m) | 1-Heptanol + 12 EO + 9 BO |
| (n) | 1-Heptanol + 3 BO + 12 EO + 1 i-BO |
| (o) | 1-Heptanol + 17 PO + 5 EO + 1 i-BO |
| (p) | 1-Heptanol + 3 BO + 12 EO + DMS |
| (q) | 1-Heptanol + 17 PO + 5 EO + DMS |
| (r) | Dipropylene glycol monobutyl ether + 3 BO + 12 EO |
| (s) | Dipropylene glycol monobutyl ether + 1 BO + 12 EO |
| (t) | Dipropylene glycol monobutyl ether + 1 PeO + 12 EO |
| (u) | Dipropylene glycol monobutyl ether + 1 DeO + 12 EO |
| (v) | Dipropylene glycol monobutyl ether + 12 EO + 1 PeO |
| (w) | Dipropylene glycol monobutyl ether + 12 EO + 1 PeO |
| (x) | Pentanol mixture + 7 BO + 12 EO |
| LeoFAT | Fatty acid methyl ester-ethoxylated |

1-26. (canceled)

27. An alkoxylated alcohol of the formula (I)
R—O—(CₘH₂ₘO)_{q}—[(C₂H₄O)_{z}—(CₙH₂ₙO)ₓ—(C₂H₄O)_{y}]_{co}—Z (I)
in which
R represents C₁-C₇-alkyl;
m represents 2 or 3;
q represents 0, 1, 2 or 3;
n represents an integer from 3 to 16;
x represents a value of from 1 to 100;
y represents a value of from 0 to 100;
z represents a value of from 0 to 100;
x+y+z corresponds to a value of from 2 to 100; and
Z represents hydrogen or an end group cap,
where at least one of y and z is greater than 0.

28. The alkoxylated alcohol of claim 27, wherein the alkoxylated alcohol is an alkoxylated alcohol of the formula (IIa)
R—O—(CₘH₂ₘO)_{q}—(CₙH₂ₙO)ₓ—(C₂H₄O)_{y}—Z (IIa),
in which y is greater than zero.

29. The alkoxylated alcohol of claim 27, wherein the alkoxylated alcohol is an alkoxylated alcohol of the formula (IIb)
R—O—(CₘH₂ₘO)_{q}—(C₂H₄O)_{z}—(CₙH₂ₙO)ₓ—Z (IIb),
in which z is greater than zero.

30. The alkoxylated alcohol of claim 27, wherein n is 3, 4, 5 or 6.

31. The alkoxylated alcohol of claim 27, wherein the sum of x, y and z, of z and x or of x and y corresponds to a value of from 10 to 30.

32. The alkoxylated alcohol of claim 27, wherein the ratio of x to z, x to y or x to (z+y) is from 1.1:1 to 25:1.

33. The alkoxylated alcohol of claim 27, wherein R is C₅-C₇-alkyl.

34. The alkoxylated alcohol of claim 27, wherein q is zero.

35. The alkoxylated alcohol of claim 27, wherein q is 1, 2 or 3.

36. The alkoxylated alcohol of claim 27, wherein m is 3.

37. The alkoxylated alcohol of claim 27, wherein Z is hydrogen.

38. The alkoxylated alcohol of claim 27, wherein Z is C₁-C₃-alkyl.

40. A composition, comprising
(a) at least one active ingredient for the treatment of plants; and
(b) at least one alkoxylated alcohol of claim 27.

41. The composition of claim 40, wherein the proportion of component (b) of the total weight of the composition is more than 10% by weight.

42. A method of using the alkoxylated alcohol of claim 27, comprising administering to a plant a composition comprising the alkoxylated alcohol of claim 27 as an adjuvant.

43. The method of claim 42, wherein the alkoxylated alcohol is an alkoxylated alcohol of the formula (IIa)
R—O—(CₘH₂ₘO)_{q}—(CₙH₂ₙO)ₓ—(C₂H₄O)_{y}—Z (IIa),
in which y is greater than zero.

44. The method of claim 42, wherein the alkoxylated alcohol is an alkoxylated alcohol of the formula (IIb)
R—O—(CₘH₂ₘO)_{q}—(C₂H₄O)_{z}—(CₙH₂ₙO)ₓ—Z (IIb),
in which z is greater than zero.

45. The method of claim 42, wherein n is 3, 4, 5 or 6.

46. The method of claim 42, wherein the sum of x, y and z, of z and x or of x and y corresponds to a value of from 10 to 30.
